(19) United States
(12) Patent Application Publication
(10) Pub. No.: US 2011/0143966 A1
(43) Pub. Date: Jun. 16, 2011

(54) Surface Modifications and Methods for their Synthesis and Use

(75) Inventors: Glenn H. McGall, Palo Alto, CA (US); Anthony D. Barone, San Jose, CA (US)

(73) Assignee: Affymetrix, INC., Santa Clara, CA (US)

(21) Appl. No.: 12/848,916

(22) Filed: Aug. 2, 2010

Related U.S. Application Data
(60) Provisional application No. 61/286,675, filed on Dec. 15, 2009.
(60) Provisional application No. 61/332,424, filed on Jul. 27, 2010.

(51) Int. Cl. C40B 50/14; C07F 7/10; C07H 21/02; C40B 50/16

(52) U.S. Cl. 506/31; 556/419; 556/418; 556/413; 506/30; 536/23.1; 546/14

(57) ABSTRACT

Novel processes are disclosed for forming an array of polymers by functionalizing the surface of particles by methods that include covalently attaching a functionalized silicon compound. Substrates such as microparticles having functionalized silicon compounds attached thereto are produced by introducing at least one carboxyl group directly by silanating a carboxylated silane compound to the surface of a microparticle. In a further aspect of the invention, the silane compound is a dipodal carboxylated silane.

### CROSS REFERENCE TO PRIOR U.S. APPLICATIONS

This application claims priority under 35 U.S.C. §119 to U.S. Provisional Application No. 61/286,675, filed Dec. 15, 2009, and U.S. Provisional Application No. 61/332,424, filed Jul. 27, 2010, the disclosures of which are incorporated by reference in their entireties for all purposes.

### BACKGROUND OF THE INVENTION

Silanating agents have been developed in the art which react with and coat surfaces, such as silica surfaces. For example, silanating agents for use in modifying silica used in high performance chromatography packings have been developed. Monofunctional silanating agents have been used to form monolayer surface coatings, while di- and tri-functional silanating agents have been used to form polymerized coatings on silica surfaces. Many silanating agents, however, produce coatings with undesirable properties including instability to hydrolysis, and the inadequate ability to mask the silica surface which may contain residual acidic silanols.

Silanating agents have been developed for the silanation of solid substrates, such as glass substrates. In some instances, these agents include functional groups that may be derivatized by a further covalent reaction. The silanating agents have been immobilized on the surface of substrates (such as glass), and used to prepare high density immobilized oligonucleotide probe arrays. For example, N-(3-(triethoxysilyl)-propyl)-4-hydroxybutyramide (Gelest Inc., Tullytown, Pa.) has been used to silanate a glass substrate prior to photochemical synthesis of arrays of oligonucleotides on the substrate, as described in McGall et al., J. Am. Chem. Soc., 119: 5081-5090 (1997), and Goldberg, et al. U.S. Pat. Nos. 5,959,098, 6,307,042, and 6,068,875; the disclosures of each are incorporated herein by reference.

Hydroxyalkylsilyl compounds have been used to prepare hydroxyalkylated substances, such as hydroxyalkylated glass substrates. N,N-Bis(hydroxyethyl)amino-propyltriethoxysilane (BHAPTES) has been used to treat glass substrates to permit the synthesis of high-density oligonucleotide arrays. McGall et al., Proc. Natl. Acad. Sci., 93: 13555-13560 (1996); Pease et al., Proc. Natl. Acad. Sci., 91: 5022-5026 (1994), and Goldberg et al. U.S. Pat. No. 5,959,098, U.S. Patent Application Publication No. 2008/0119371, U.S. Patent Application Publication No. 2005/0080284, the disclosures of which are incorporated herein by reference in their entireties. Acetoxypropyltriethoxy-silane has been used to treat glass substrates to prepare them for oligonucleotide array synthesis, as described in PCT Publication No. 97/39151, the disclosure of which is incorporated herein by reference. 3-Glycidoxypropyltrimethoxysilane has been used to treat a glass support to provide a linker for the synthesis of oligonucleotides (See EP Patent Application No. 89120696.3, the disclosure of which is incorporated herein by reference in its entirety for all purposes).

Methods have been developed in the art for stabilizing surface bonded silicon compounds. The use of sterically hindered silanating agents is described in Kirkland et al., Anal. Chem. 61: 2-11 (1989); and Schneider et al., Synthesis, 1027-1031 (1990). However, the use of these surface bonded silanating agents is disadvantageous, because they typically require forcing conditions to achieve bonding to the glass, since their hindered nature makes them less reactive with the substrate.

The invention addresses this and other needs by providing, in one embodiment, functionalized silicon compounds that have derivatizable functional groups that can be used to form functionalized coatings on materials and substrates, such as glass. In a further embodiment, functionalized silicon compounds are provided that can be used to form coatings on materials that are stable under the conditions of use.

### SUMMARY OF THE INVENTION

In one embodiment, functionalized silicon compounds and methods for their use are provided. The functionalized silicon compounds, in one embodiment, each include an activated silicon group and a derivatizable functional group. Exemplary derivatizable functional groups include, but are not limited to, halogen, hydroxy, thiol, amine, hydrazine, aminooxy, sulfonate, sulfate, azide, carbonyl, carboxyl, carboxylate, thiocarboxyl, aldehyde, alkene, alkyne, disulfide, isocyanate, isothiocyanate, as well as modified forms thereof, such as activated or protected forms. The functionalized silicon compounds may be covalently attached to surfaces to form functionalized surfaces which may be used in a wide range of different applications, for example, functionalized surfaces for microarray applications.

In one embodiment, the silicon compounds are attached to the surface of a substrate comprising silica, such as a glass substrate, to provide a functionalized surface on the silica containing substrate, to which molecules, including polypeptides and polynucleotides, may be attached. In one embodiment, after the covalent attachment of a functionalized silicon compound to the surface of a solid silica substrate to form a functionalized coating on the substrate, an array of nucleic acids may be covalently attached to the substrate through the functionalized coating. Thus, in one embodiment, the methods permit the formation of high density arrays of nucleic acids immobilized on a substrate, which may be used in conducting high volume nucleic acid hybridization assays.

According to one embodiment, a method of forming an array of nucleic acids is provided. The method comprises silanating a surface of a substrate by covalently attaching a plurality of functionalized silicon compounds to the substrate. During the silanation step, in one embodiment, at least one carboxyl group is directly introduced by silanating the surface of the substrate with a carboxylated silane compound. After the surface of the substrate is silanated, biological polymers are conjugated to the carboxylated silane. In a further embodiment, the method is used to silanate the surface of a microparticle substrate. In a further embodiment, the microparticle is a magnetic bead.

In one embodiment, a silane-functionalized compound represented by Formula 1 is provided.

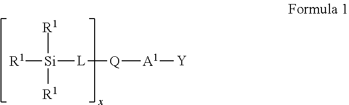

wherein, x is an integer selected from 1 to 3,

each occurrence of R¹ is independently any alkoxy, aryloxy or halogen or is a lower alkyl where at least one of the R¹ groups is an alkoxy or halogen,

each occurrence of L is independently a spacer group (e.g., an aliphatic chain having at least two carbon atoms) optionally comprising one or more organofunctional moieties comprising a functional group selected from the group consisting of ether, amine, sulfide, sulfoxyl, carbonyl, thione, ester, thioester, carbonate, thiocarbonate, carbamate, thiocarbamate, amide, thioamide, urea and thiourea groups, and

Q is N, C₁-C₁₀ alkyl or C₁-C₁₀ substituted alkyl,

A¹ is a linking group comprising a straight chain alkyl, branched alkyl, cycloalkyl, alkenyl, alkynyl, aryl or heteroaryl. A¹ optionally comprises one or more organofunctional moieties selected from the group consisting of ether, amine, sulfide, sulfonyl, sulfate, carbonyl, thione, ester, thioester, carbonate, thiocarbonate, carbamate, thiocarbamate, amide, thioamide, urea and thiourea groups.

Y is a derivatizable functional group or protected functional group selected from the group consisting of halogen, hydroxy, thiol, amine, hydrazine, aminooxy, sulfonate, sulfate, azide, carbonyl, carboxyl, carboxylate, thiocarboxyl, aldehyde, alkene, alkyne, disulfide, isocyanate, isothiocyanate or modified forms thereof.

In one embodiment, x is 2, and the compound of Formula 1 has the structure of Formula 1(A):

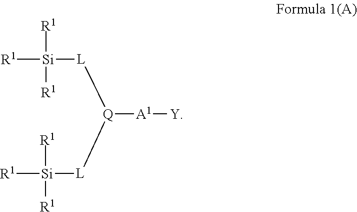

In one embodiment of compounds of Formula 1, x is 2 and Q is N. In another embodiment, x is 3 and Q is C₁-C₁₀ alkyl or C₁-C₁₀ substituted alkyl. In a further embodiment, Q is methyl.

In one embodiment, Q is methyl, ethyl or propyl. In one embodiment, if x is 3, then Q is methyl.

In one embodiment, at least one occurrence of L is an aliphatic chain comprising at least 2 atoms.

In another embodiment of the compounds of Formula 1, one or more of the R¹ moities are reacted with a surface to provide a surface of Formula 2 or 2(A).

In one embodiment of Formula 1(A), A¹ is a straight chain alkyl. In a further embodiment of Formula 1(A), A¹ comprises one or more organofunctional moieties. In yet a further embodiment of Formula 1(A), A¹ is a C₃, C₄, C₅, C₆, C₇, C₈, C₉ or C₁₀ straight chain alkyl. In even a further embodiment of Formula 1(A), A¹ comprises a carboxyl group.

In one embodiment of Formula 1(A), each L group is a carbon chain having 3, 4, or 5 carbon atoms, Q is N, and A¹ and Y together, form the group

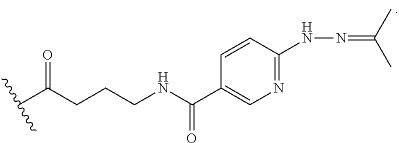

For example, in one embodiment, A¹ is —C(═O)CH₂CH₂NHC(═O)— and Y is 2-(2-(propan-2-ylidene)hydrazinyl)pyridine. In one embodiment, this compound can be attached to a substrate, for example, a microparticle. In a further embodiment, a plurality of compounds are attached to one or more microparticles.

In another Formula I(A) embodiment, each L group is a carbon chain having 3, 4, or 5 carbon atoms, Q is N, and A¹ and Y together, form the group

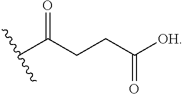

For example, in one embodiment, A¹ is a C₃ straight chain alkyl comprising a carboxyl moiety (—C(═O)CH₂CH₂—) and Y is COOH. In a further embodiment, each L group has 3 carbons. In one embodiment, this compound can be attached to a substrate, for example, a microparticle. In a further embodiment, a plurality of compounds are attached to one or more microparticles.

In one embodiment, a compound of Formula 1, or a plurality of compounds of Formula 1, can be covalently attached to a surface, to form a modified surface of Formula 2 or 2(A):

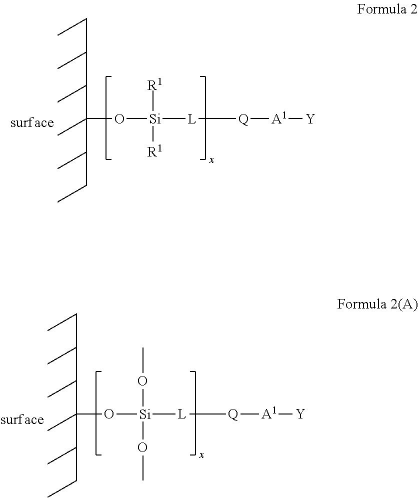

wherein, R¹, x, Q, A¹ and Y are defined as provided in Formula 1.

In one embodiment of Formula 2 or 2(A), at least one occurrence of L is an aliphatic chain comprising at least two carbon atoms. In another embodiment, the modified surface of Formula 2, is provided by the attachment of at least two R¹ moieties of the compound of Formula 1 to the surface.

In another embodiment of Formula 2 or 2(A), Q is N, methyl or ethyl.

In yet another embodiment of Formula 2, x is 2.

A¹, in one Formula 2, embodiment, is methyl, ethyl or a 6 or 5 carbon cycloalkyl.

### BRIEF DESCRIPTION OF THE FIGURES

FIG. 1 illustrates a reaction of an electrophilic glass surface with a nucleophilic oligonucleotide modification.

FIG. 2 illustrates a reaction of a glass support coated with milder electrophilic functional groups.

FIG. 3 illustrates a reaction of a surface aldehyde with an alkylamine-modified oligonucleotide.

FIG. 4 illustrates a reaction of a surface aldehyde with a hydrazino-modified oligonucleotide.

FIG. 5 illustrates reagents for introducing aldehyde groups. FIGS. 5A and 5B illustrate reagents for introducing aldehyde groups. FIG. 5C illustrates a reagent for producing a dipodal hydrazone according to an embodiment.

FIGS. 6A and 6B show reagents for introducing hydrazine groups.

FIGS. 7A and 7B illustrate a reaction of a surface hydrazine with an adehyde-modified oligonucleotide according to another embodiment.

FIGS. 8A and 8B illustrate reagents for introducing aldehyde groups according to a further embodiment.

FIGS. 9A and 9B illustrate reagents for introducing hydrazine groups. FIG. 9C illustrates a reagent for producing a dipodal hydrazone according to an embodiment.

FIGS. 10A and 10B illustrate general schemes 10-1 and 10-2 according to an embodiment.

FIGS. 11A-11E illustrate examples of reactions from formulae illustrated in FIG. 10A according to one embodiment.

FIGS. 12A-12C illustrate examples of reactions from formulae illustrated in FIG. 10B according to an embodiment.

FIG. 13 illustrates scheme 13-1 for introducing surface carboxyl groups in two steps.

FIG. 14 illustrates scheme 13-2 of directly introducing carboxyl groups at the silanation step according to an embodiment.

FIG. 15 illustrates scheme 13-3 of preparing carboxylated silanes according to an embodiment.

FIGS. 16A and 16B illustrate encoded particles. FIG. 16A illustrates a schematic of individual encoded particles. FIG. 16B illustrates SEM images of the surface of an encoded particle.

FIG. 17 illustrates a schematic of work-flow for processing printed microparticles.

FIG. 18 illustrates the synthesis of 6-(N′-isopropylidene-hydrazino)-nicotinic acid, N-hydroxysuccinimidyl ester from 6-hydrazino-nicotinic acid.

FIG. 19 illustrates the synthesis of bis-(trimethoxysilylpropyl)-6-(N′-isopropyl-idene-hydrazino)-nicotinicamide (6-hydrazino-N,N-bis-(3-trimethoxysilylpropyl)nicotinamide silane XV).

FIG. 20 illustrates the synthesis of N-trimethoxysilylpropyl-(4-N′-isopropyl-idene-hydrazino)-benzamide from 4-(N′-Isopropylidene-hydrazino)-benzoic acid N-hydroxysuccinimidyl ester.

FIG. 21 shows one embodiment of a silanation and oligonucleotide coupling of microparticles.

FIG. 22 illustrates a silanation and oligonucleotide coupling of DNA to particles.

FIG. 23 illustrates the kinetics of hydrazine formation at 1 μM oligo concentration as a function of coupling pH, presence of catalyst and time for deprotection of isopropylidine protecting group.

FIG. 24 illustrates the kinetics of hydrazine formation as a function of oligonucleotide concentration.

FIG. 25 illustrates a scheme of attaching an oligonucleotide possessing a cleavable fluorescent tag which can be quantified by HPLC.

FIG. 26 illustrates fluorescence intensity results as a function of oligonucleotide coupling concentration.

FIG. 27A illustrates HPLC density versus oligo coupling concentration. FIG. 27B illustrates HPLC density versus scan results.

FIG. 28 illustrates intensity hybridization intensity of 20 nM complimentary CY3-labeled target at 40° C. for 2 hours versus oligo coupling concentration.

FIG. 29 illustrates a typical ion-exchange chromatogram of a density measurement from the cleavage of about 2×10⁵ particles.

FIGS. 30A and 30B illustrate scanned images. FIG. 30A illustrates a typical image of a mixture of fluorescein-labeled DNA conjugated particles and bare particles scanned in the reflectance mode. FIG. 30B illustrates an image of fluorescein-labeled DNA conjugated particles in FIG. 30A scanned in the fluorescence mode.

FIGS. 31A and 31B illustrate images of hybridization of particles. FIG. 31A illustrates an image with a Cy3-labeled complimentary target sequence. FIG. 31B illustrates an image with a Cy3-labeled non-complimentary sequence.

FIG. 32 is a graph showing fluorescence intensity results.

FIG. 33 is a graph showing fluorescence intensity results.

FIG. 34 illustrates a typical HPLC chromatogram of denatured targed from the particles.

FIG. 35 illustrates an accelerated thermal stability study of conjugated particles.

FIG. 36 illustrates kinetic relative rate curves for particles versus chip (planar glass).

### DETAILED DESCRIPTION OF THE INVENTION

Although the invention is described in conjunction with the exemplary embodiments, the invention is not limited to these embodiments. On the contrary, the invention encompasses alternatives, modifications and equivalents, which may be included within the spirit and scope of the invention.

The invention relates to diverse fields impacted by the nature of molecular interaction, including chemistry, biology, medicine and diagnostics. Methods disclosed herein are advantageous in fields, such as those in which genetic information is required quickly, as in clinical diagnostic laboratories or in large-scale undertakings such as systems biology inquiries and full organism DNA sequencing, for example, the Human Genome Project.

The specification references and incorporates the disclosures of patents, patent applications and other references for details known to those of ordinary skill in the art. Therefore, when a patent, application, or other reference is cited herein, it should be understood that the entire disclosure of the document cited is incorporated by reference in its entirety for all purposes as well as for the proposition that is recited. All documents, e.g., publications and patent applications, cited in this disclosure, including the foregoing, are incorporated herein by reference in their entireties for all purposes to the same extent as if each of the individual documents were specifically and individually indicated to be so incorporated herein by reference in its entirety.

Throughout this disclosure, various aspects of this invention can be presented in a range format. It should be understood that when a description is provided in range format, this is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range.

The invention may employ, unless otherwise indicated, conventional techniques and descriptions of organic chemistry, polymer technology, molecular biology (including recombinant techniques), cell biology, biochemistry, and immunology, which are within the skill of one of ordinary skill in the art. Such conventional techniques include polymer array synthesis, hybridization, ligation, and detection of hybridization using a detectable label (e.g., a fluorescent label). Specific illustrations of suitable techniques are provided by reference to the examples, provided below. However, other equivalent conventional procedures may also be employed. Such conventional techniques and descriptions may be found in standard laboratory manuals, such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995), Biochemistry, 4th Ed., Freeman, N.Y., Gait, Oligonucleotide Synthesis: A Practical Approach, (1984), IRL Press, London, Nelson and Cox (2000), Lehninger, Principles of Biochemistry, 3^{rd} Ed., W. H. Freeman Pub., New York, N.Y., and Berg et al. (2002), Biochemistry, 5^{th} Ed., W. H. Freeman Pub., New York, N.Y., all of which are herein incorporated in their entirety by reference for all purposes.

### DEFINITIONS

As used in this application, the singular form “a,” “an”, and “the” include plural references unless the context clearly dictates otherwise. For example, the term “an agent” includes a plurality of agents, including mixtures thereof.

The term “array” as used herein refers to an intentionally created collection of molecules which can be prepared either synthetically or biosynthetically. The molecules in the array can be identical or different from each other. The array can assume a variety of formats, including, but not limited to, libraries of soluble molecules, and libraries of compounds tethered to resin beads, silica chips, or other solid supports. An array may include polymers of a give length having all possible monomer sequences made up of a specific bases set of monomers, or a specific subset of such an array. In other cases as array may be formed from inorganic materials (See Schultz et al., PCT Publication No. WO 96/11878).

The term “functional group” as used herein refers to a reactive chemical moiety present on a given monomer, polymer or substrate surface. Examples of functional groups include, e.g., the 3′ and 5′ hydroxyl groups of nucleotides and nucleosides, as well as the reactive groups on the nucleobases of the nucleic acid monomers, e.g., the exocyclic amine group of guanosine, as well as amino and carboxyl groups on amino acid monomers.

Functionalized silicon compounds are provided, as well as methods for their synthesis and use. The functionalized silicon compounds may be used to form functionalized coatings on a variety of surfaces such as the surfaces of glass substrates. A variety of functionalized silicon compounds, which are available commercially, or which may be synthesized as disclosed herein, may be used in the methods disclosed herein to react with surfaces to form functionalized surfaces which may be used in a wide range of different applications.

In one embodiment, the functionalized silicon compounds are covalently attached to a substrate surface, to produce a functionalized substrate surface. For example, the silicon compounds of the invention may be attached to the surfaces of glass substrates, to provide a functionalized glass surface to which molecules, including polypeptides and nucleic acids, may be attached.

As used herein, the term “silicon compound” refers to a compound comprising at least one silicon atom. In one embodiment, the silicon compound is a silanating agent comprising an activated silicon group, wherein the activated silicon group comprises a silicon atom covalently linked to at least one reactive group, such as an alkoxy or halide, such that the silicon group is capable of reacting with a functional group, for example on a surface of a substrate, to form a covalent bond with the surface. For example, the activated silicon group on the silicon compound can react with the surface of a silica substrate comprising surface Si—OH groups to create siloxane bonds between the silicon compound and the silica substrate. Exemplary activated silicon groups include —Si(OMe)₃; —SiMe(OMe)₂; —SiMeCl₂; —SiMe(OEt)₂; —SiCl₃ and —Si(OEt)₃; —Si—NMe₂, —Si—NEt₂, —Si—N(SiMe₃)₂.

As used herein, the term “functionalized silicon compound” refers to a silicon compound comprising a silicon atom and a derivatizable functional group. In one embodiment, the functionalized silicon compound is a functionalized silanating agent and includes an activated silicon group and a derivatizable functional group.

As used herein, the term “derivatizable functional group” refers to a functional group that is capable of reacting to permit the formation of a covalent bond between the silicon compound and another substance, such as a biopolymer. Exemplary derivatizable functional groups include, but are not limited to, hydroxy, thiol, amine, hydrazine, aminooxy, sulfonate, sulfate, azide, carbonyl, carboxyl, carboxylate, thiocarboxyl, aldehyde, alkene, alkyne, disulfide, isocyanate, isothiocyanate, as well as modified forms and analogues thereof, such as activated or protected forms. The term “activated” refers to derivatives of the indicated group that are synthetically equivalent (synthons) but are more reactive than the unactivated group. The term “protected” generally refers to easily formed derivatives of the indicated group which prevent reaction of the group under certain conditions, and which can subsequently be converted back to the unprotected group to allow reaction when desired. Activated and protected forms of the indicated derivatizable functional groups are known in the art.

Derivatizable functional groups also include substitutable leaving groups such as halogen or sulfonyloxy. In one embodiment, the derivatizable functional group is a group, such as a hydroxyl group, that is capable of reacting with activated nucleotides to permit nucleic acid synthesis. For example, the functionalized silicon compound may be covalently attached to the surface of a substrate, such as glass, and then derivatizable hydroxyl groups on the silicon compound may be reacted with an activated phosphate group on a protected nucleotide phosphoramidite or H-phosphonate, and then stepwise addition of further protected nucleotide phosphoramidites or H-phosphonates can result in the formation of a nucleic acid covalently attached to the support. The nucleic acids also may be attached to the derivatizable group via a linker. In a further embodiment, arrays of nucleic acids may be formed covalently attached to the substrate which are useful in conducting nucleic acid hybridization assays.

The term “monomer/building block” as used herein refers to a member of the set of smaller molecules which can be joined together to form a larger molecule or polymer. The set of monomers includes but is not restricted to, for example, the set of common L-amino acids, the set of D-amino acids, the set of natural or synthetic amino acids, the set of nucleotides (both ribonucleotides and deoxyribonucleotides, natural and unnatural) and the set of pentoses and hexoses. As used herein, monomer refers to any member of a basis set for synthesis of a larger molecule. A selected set of monomers forms a basis set of monomers. For example, the basis set of nucleotides includes A, T (or U), G and C. In another example, dimers of the 20 naturally occurring L-amino acids form a basis set of 400 monomers for synthesis of polypeptides. Different basis sets of monomers may be used in any of the successive steps in the synthesis of a polymer. Furthermore, each of the sets may include protected members which are modified after synthesis.

The terms “oligonucleotide”, “polynucleotide” and “nucleic acid” as used herein, refer to a polymeric form of nucleotides of any length, either ribonucleotides or deoxyribonucleotides (or a combination thereof), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components.

As used herein, the terms “oligonucleotide”, “polynucleotide” and “nucleic acid” are synonymous, and refer to a nucleic acid polymer having at least 2, at least 3, at least 4, at least 5, at least 6, at least 7, at least 8, at least 10, at least 12, at least 14, at least 15, at least 16, at least 18, or at least 20 nucleotides in length. Alternatively or additionally, “oligonucleotide” and “polynucleotide” refer to a compound that specifically hybridizes to a polynucleotide. Polynucleotides of the invention include sequences of deoxyribonucleic acid (DNA) or ribonucleic acid (RNA) which may be isolated from natural sources, recombinantly produced or artificially synthesized and mimetics thereof. A further example of a polynucleotide of the invention may be peptide nucleic acid (PNA). The invention also encompasses situations in which there is a nontraditional base pairing such as Hoogsteen base pairing which has been identified in certain tRNA molecules and postulated to exist in a triple helix.

The term “probe” as used herein, refers to a surface-immobilized or free-in-solution molecule that can be recognized by a particular target. U.S. Pat. No. 6,582,908 provides an example of arrays having all possible combinations of nucleic acid-based probes having a length of 10 bases, and 12 bases or more. In one embodiment, a probe may consist of an open circle molecule, comprising a nucleic acid having left and right arms whose sequences are complementary to the target, and separated by a linker region. Open circle probes are described in, for instance, U.S. Pat. No. 6,858,412, and Hardenbol et al., Nat. Biotechnol., 21(6):673 (2003). In another embodiment, a probe, such as a nucleic acid, may be attached to a microparticle carrying a distinguishable code. Examples of encoded microparticles, methods of making the same, methods for fabricating the microparticles, methods and systems for detecting microparticles, and the methods and systems for using microparticles are described in U.S. Patent Application Publication Nos. 2008/0038559, 2007/0148599, and PCT Publication No. WO 2007/081410, each of which is hereby incorporated by reference in its entirety. Such microparticles are preferably encoded such that the identity of a probe borne by a microparticle can be read from a distinguishable code. The code can be in the form of a tag, which may itself be a probe, such as an oligonucleotide, a detectable label, such as a fluorophore, or embedded in the microparticle, for example, as a bar code. Microparticles bearing different probes have different codes. Microparticles are typically distributed on a support by a sorting process in which a collection of microparticles are placed on the support and the microparticles distributed on the support. The location of the microparticles after distribution on the support can be defined by indentations such as wells or by association to adhesive regions on the support, among other methods. The microparticles may be touching or they may be separated so that individual microparticles are not touching.

Examples of nucleic acid probe sequences that may be investigated by this invention include, but are not restricted to, those that are complementary to genes encoding agonists and antagonists for cell membrane receptors, toxins and venoms, viral epitopes, hormones (for example, opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

As used herein, the term “protecting group” refers to a material which is chemically bound to a reactive functional group on a monomer unit or polymer and which protective group may be removed upon selective exposure to an activator such as a chemical activator, or another activator, such as electromagnetic radiation or light, especially ultraviolet and visible light. Protecting groups that are removable upon exposure to electromagnetic radiation, and in particular light, are termed “photolabile protecting groups”. Examples of suitable protecting groups include those described in “Protecting Groups”, P. Kocienski, 3^{rd} Ed., Georg Thieme Verlag or “Protecting Groups in Organic Synthesis”, T. W. Greene and P. G. M. Wuts, 3^{rd} Ed., John Wiley & Sons. Examples of suitable photolabile protecting groups include those described in “Handbook of Synthetic Photochemistry, A. Albini, M. Fagnono (Eds.), Wiley-VCH., and U.S. patent application Ser. No. 12/510,501 and U.S. Publication Nos. 20050101765 and 20030040618, each of which is incorporated by reference in its entirety for all purposes.

The term “alkyl,” as a group, refers to a straight or branched hydrocarbon chain containing the specified number of carbon atoms. When the term “alkyl” is used without reference to a number of carbon atoms, it is to be understood to refer to a C₁-C₁₀ alkyl. For example, C₁₋₁₀ alkyl refers to a straight or branched alkyl containing at least 1, and at most 10, carbon atoms. Examples of “alkyl” as used herein include, but are not limited to, methyl, ethyl, n-propyl, n-butyl, n-pentyl, isobutyl, isopropyl, t-butyl, hexyl, heptyl, octyl, nonyl and decyl.

The term “substituted alkyl” as used herein denotes alkyl radicals wherein at least one hydrogen is replaced by one more substituents such as, but not limited to, hydroxy, alkoxy, aryl (for example, phenyl), heterocycle, halogen, trifluoromethyl, pentafluoroethyl, cyano, cyanomethyl, nitro, amino, amide (e.g., —C(O)NH—R where R is an alkyl such as methyl), amidine, amido (e.g., —NHC(O)—R where R is an alkyl such as methyl), carboxamide, carbamate, carbonate, ester, alkoxyester (e.g., —C(O)O—R where R is an alkyl such as methyl) and acyloxyester (e.g., —OC(O)—R where R is an alkyl such as methyl), or two hydrogens on a single carbon is replaced with oxygen to provide a carbonyl group. The definition pertains whether the term is applied to a substituent itself or to a substituent of a substituent.

The term “cycloalkyl” group as used herein refers to a non-aromatic monocyclic hydrocarbon ring of 3 to 8 carbon atoms such as, for example, cyclopropyl, cyclobutyl, cyclopentyl, cyclohexyl or cycloheptyl.

The term “substituted cycloalkyl” as used herein denotes a cycloalkyl group further bearing one or more substituents as set forth herein, such as, but not limited to, hydroxy, alkoxy, aryl (for example, phenyl), heterocycle, halogen, trifluoromethyl, pentafluoroethyl, cyano, cyanomethyl, nitro, amino, amide (e.g., —C(O)NH—R where R is an alkyl such as methyl), amidine, amido (e.g., —NHC(O)—R where R is an alkyl such as methyl), carboxamide, carbamate, carbonate, ester, alkoxyester (e.g., —C(O)O—R where R is an alkyl such as methyl) and acyloxyester (e.g., —OC(O)—R where R is an alkyl such as methyl), or two hydrogens on a single carbon is replaced with oxygen to provide a carbonyl group. The definition pertains whether the term is applied to a substituent itself or to a substituent of a substituent.

As used herein, the terms “solid support”, “support”, and “substrate” are synonymous, and refer to a material or group of materials having a rigid or semi-rigid surface or surfaces. In one embodiment, at least one surface of the solid support is substantially flat. In one embodiment, regions of the substrate are separated by non-flat areas, for example, wells, trenches, grooves, raised regions, pins, etched trenches and the like. It may be desirable to physically separate synthesis regions for different compounds with the aforementioned structures.

Solid supports include any of a variety of fixed organizational support matrices. According to some embodiments, the solid support(s) is in the form of slides, solid chips, beads, resins, gels, microspheres, microparticles or other geometric configurations. U.S. Pat. No. 5,744,305, incorporated herein by reference, provides examples of substrates/solid supports.

In another embodiment, the solid support may be, for example, biological, nonbiological, organic, inorganic, or a combination thereof. In one embodiment, a solid support is in the form of particles, microparticles, strands, gels, sheets, tubing, spheres, containers, capillaries, pads, slices, films, plates, and slides. Depending upon the intended end use of the solid support, one of ordinary skill in the art will readily know how to go about selecting the appropriate geometric shape and material.

The term “target” as used herein, refers to a molecule that has an affinity for a given probe. Targets may be naturally-occurring or synthetic molecules. Also, they can be employed in their unaltered state or as aggregates with other species. Targets may be attached, covalently or noncovalently, to a binding member, either directly or via a specific binding substance. Examples of targets include, but are not restricted to, antibodies or fragments thereof, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term target is used herein, no difference in meaning is intended between these two terms. A “Probe Target Pair” is formed when two macromolecules have combined through molecular recognition to form a complex.

The term “wafer” as used herein, refers to a substrate having surface to which a plurality of microarrays can be bound or synthesized. In one embodiment, a “wafer” is a substantially flat substrate from which a plurality of individual arrays or chips may be fabricated.

The term “array” or “chip” is used to refer to the final product of the individual array of polymer sequences, having a plurality of different positionally distinct polymer sequences coupled to the surface of the substrate. The size of a substrate wafer is generally defined by the number and nature of arrays that will be produced from the wafer. For example, more complex arrays, e.g., arrays having all possible polymer sequences produced from a basis set of monomers and having a given length, will generally utilize larger areas and thus employ larger substrates, whereas simpler arrays may employ smaller surface areas, and thus, less substrate.

### Compounds

In one embodiment, functionalized silicon compounds and methods for their use are provided. The functionalized silicon compounds each include an activated silicon group and a derivatizable functional group. Exemplary non-limiting derivatizable functional groups include halogen, hydroxy, thiol, amine, hydrazine, aminooxy, sulfonate, sulfate, azide, carbonyl, carboxyl, carboxylate, thiocarboxyl, aldehyde, alkene, alkyne, disulfide, isocyanate, isothiocyanate, as well as modified forms thereof, such as activated or protected forms. The functionalized silicon compounds may be covalently attached to a surface to form a functionalized surface which may be used in a wide range of different applications. In one embodiment, the silicon compounds are attached to the surface of a substrate comprising silica, such as a microparticle, to provide a functionalized surface on the silica containing substrate, to which molecules, including polypeptides and polynucleotides, may be attached.

In one embodiment, after covalent attachment of a plurality of functionalized silicon compounds to the surface of a solid silica substrate to form a functionalized coating on the substrate, an array of nucleic acids may be covalently attached to the substrate through the functionalized coating. Thus, the method provided herein permits the formation of high density arrays of nucleic acids immobilized on a substrate, which may be used in conducting high volume nucleic acid hybridization assays.

### Substrate Preparation

The term “substrate” refers to a material having a rigid or semi-rigid surface onto which the polymers are placed, attached or bound, for example. In some embodiments, at least one surface of the substrate will be substantially flat or planar, although in some embodiments, it may be desirable to physically separate synthesis regions for different polymers with, for example, wells, raised regions, etched trenches, or the like. According to one embodiment, small beads are provided on the surface which are released upon completion of the synthesis. In one embodiment, substrates comprise planar crystalline substrates such as silica based substrates (e.g., glass, quartz, or the like), or crystalline substrates used in, e.g., the semiconductor and microprocessor industries, such as silicon, gallium arsenide and the like. These substrates are generally resistant to the variety of synthesis and analysis conditions to which they may be subjected. In one embodiment, substrates are transparent to allow the photolithographic exposure of the substrate from either direction, for example, see U.S. Pat. No. 5,143,854, incorporated by reference in its entirety for all purposes. In another embodiment, the substrate is a microparticle or a plurality of microparticles. Examples of encoded microparticles, methods of making the same, methods for fabricating the microparticles, methods and systems for detecting microparticles, and the methods and systems for using microparticles are described in U.S. Patent Application Publication Nos. 2008/0038559, 2007/0148599, and PCT Application No. WO 2007/081410, each of which is hereby incorporated by reference in its entirety for all purposes.

Silica aerogels may also be used as a substrate or portion of a substrate. Silica aerogel substrates may be used as free standing substrates or as a surface coating for another rigid substrate. Aerogel substrates provide the advantage of large surface area for polymer synthesis, e.g., 400 to 1000 m²/gm, or a total useful surface area of 100 to 1000 cm² for a 1 cm² piece of aerogel substrate. Such aerogel substrates may generally be prepared by methods known in the art. For example, in one embodiment, a silica aerogel substrate is prepared by the base catalyzed polymerization of (MeO)₄Si or (EtO)₄Si in ethanol/water solution at room temperature. Porosity may be adjusted by altering reaction conditions, by methods known in the art.

In one embodiment, the substrate wafer ranges in size of from about 1″×about 1″ to about 12″×about 12″, and will have a thickness of from about 0.5 mm to about 5 mm. Individual substrate segments which include the individual arrays, or in some cases a desired collection of arrays, are typically much smaller than the wafers, measuring from about 0.2 cm×about 0.2 cm to about 5 cm×about 5 cm. In particular aspects, the substrate wafer is about 5″×about 5″ whereas the substrate segment is approximately 1.28 cm×1.28 cm. Although a wafer can be used to fabricate a single large substrate segment, typically, a large number of substrate segments will be prepared from a single wafer. For example, a wafer that is 5″×5″ can be used to fabricate upwards of 49 separate 1.28 cm×1.28 cm substrate segments. The number of segments prepared from a single wafer will generally vary depending upon the complexity of the array, and the desired feature size.

Although primarily described in terms of flat or planar substrates, the invention may also be practiced with substrates having substantially different conformations. For example, in one embodiment, the invention pertains to substrates that exist as particles, microparticles, strands, precipitates, gels, sheets, tubing, spheres, containers, capillaries, pads, slices, films, plates, slides, etc. In another embodiment, the substrate is a glass tube or microcapillary. The microcapillary substrate provides advantages of higher surface area to volume ratios, reducing the amount of reagents necessary for synthesis. Similarly, the higher surface to volume ratio of these capillary substrates imparts more efficient thermal transfer properties.

In one embodiment, preparation of the polymer arrays is simplified through the use of capillary based substrates. For example, minimizing differences between the regions on the array, or “cells”, and their “neighboring cells” is simplified in that there are only two neighboring cells for any given cell (see discussion below for edge minimization in chip design). Spatial separation of two neighboring cells on an array merely involves the incorporation of a single blank cell, as opposed to full blank lanes as generally used in a flat substrate conformation. This substantially conserves the surface area available for polymer synthesis. Manufacturing design may also be simplified by the linear nature of the substrate. In particular, the linear substrate may be moved down a single mask in a direction perpendicular to the length of the capillary. As it is moved, the capillary encounters linear reticles (translucent regions of the mask), one at a time, thereby exposing selected regions within the capillary or capillary. This can allow bundling of parallel capillaries during synthesis wherein the capillaries are exposed to thicker linear reticles, simultaneously, for a batch processing mode, or individual capillaries may be placed on a mask having all of the linear reticles lined up so that the capillary can be stepped down the mask in one direction. Subsequent capillaries may be stepped down the mask at least one step behind the previous capillary. This employs an assembly line structure to the substrate preparation process.

### Silanation of Substrates

The invention provides silanated substrate surfaces (surfaces treated with functionalized silanes as described herein) with additional derivatization sites. In one embodiment, a substrate surface is derivatized with a plurality of silane functionalized compounds to provide sites or functional groups on the substrate surface for synthesizing the various polymer sequences (e.g., polynucleotides) on that surface. In particular, derivatization provides reactive functional groups, e.g., hydroxyl, carboxyl, amino groups or the like, to which the first monomers in the polymer sequence may be attached. In one embodiment, the substrate surface is derivatized using a silane functionalized compound in either water or ethanol. In another embodiment, the surface is coated and derivatized by contacting the coated surface with a solution of a silanation reagent. In a further embodiment, the contacting of the surface of the substrate with the silanation reagent is carried out by controlled vapor deposition of the silanation reagent on the surface.

Silanation reagents have been developed in the art which react with and coat surfaces, such as silica surfaces. For example, silanation reagents for use in modifying silica used in high performance chromatography packings have been developed. Monofunctional silanation reagents have been used to form monolayer surface coatings, while di- and tri-functional silanation reagents have been used to form polymerized coatings on silica surfaces.

Many silanation reagents, however, produce coatings with undesirable properties including instability to hydrolysis and the inadequate ability to mask the silica surface which may contain residual acidic silanols. See U.S. Pat. No. 6,262,216 and U.S. Patent Application Publication No. U.S. 2001/0021506, both of which are incorporated by reference.

Silanation reagents have been developed for the silanation of solid substrates, such as glass substrates, that include functional groups that may be derivatized by further covalent reaction. The silanation reagents have been immobilized on the surface of substrates, such as glass, and used to prepare high density immobilized oligonucleotide arrays. For example, N-(3-(triethoxysilyl)-propyl)-4-hydroxybutyramide (PCR Inc., Gainesville, Fla. and Gelest Inc., Tullytown, Pa.) has been used to silylate a glass substrate prior to photochemical synthesis of arrays of oligonucleotides on the substrate, as described in McGall et al., J. Am. Chem. Soc., 119: 5081-5090 (1997), the disclosure of which is incorporated herein by reference.

Hydroxyalkylsilyl compounds have been used to prepare hydroxyalkylated substances, such as glass substrates. N,N-Bis-(hydroxyethyl)aminopropyl-triethoxysilane (BHAPTES) has been used to treat glass substrates to permit the synthesis of high-density oligonucleotide arrays. McGall et al., Proc. Natl. Acad. Sci., 93: 13555-13560 (1996); and Pease et al., Proc. Natl. Acad. Sci., 91: 5022-5026 (1994), the disclosures of which are incorporated herein. Acetoxypropyl-triethoxysilane has been used to treat glass substrates to prepare them for oligonucleotide array synthesis, as described in PCT WO 97/39151, incorporated herein by reference. 3-Glycidoxy propyltrimethoxysilane has been used to treat a glass substrate to provide a linker for the synthesis of oligonucleotides (EP Patent Application No. 89120696.3).

Methods have been developed in the art for stabilizing surface bonded silicon compounds. The use of sterically hindered silanation reagents is described in Kirkland et al., Anal. Chem. 61: 2-11 (1989); and Schneider et al., Synthesis, 1027-1031 (1990). However, the use of these surface bonded silanation reagents is disadvantageous, because they typically require very forcing conditions to achieve bonding to the glass, since their hindered nature makes them less reactive with the substrate.

Additionally, silanes can be prepared having protected or “masked” hydroxyl groups and which possess significant volatility. As such, these silanes can be readily purified by, e.g., distillation, and can be readily employed in gas-phase deposition methods of silanating substrate surfaces. After coating these silanes onto the surface of the substrate, the hydroxyl groups may be deprotected with a brief chemical treatment, e.g., dilute acid or base, which will not attack the substrate-silane bond, so that the substrate can then be used for polymer synthesis. Examples of such silanes include acetoxyalkylsilanes, such as acetoxyethyltrichlorosilane and acetoxypropyltrimethoxysilane, which may be deprotected after application using, e.g., vapor phase ammonia and methylamine or liquid phase aqueous or ethanolic ammonia and alkylamines. Epoxyalkylsilanes may also be used, such as glycidoxypropyltrimethoxysilane which may be deprotected using, e.g., vapor phase HCl, trifluoroacetic acid or the like, or liquid phase dilute HCl.

The physical operation of silanation of the substrate generally involves dipping or otherwise immersing the substrate in the silane solution. Following immersion, the substrate is generally spun as described for the substrate stripping process, e.g., laterally, to provide a uniform distribution of the silane solution across the surface of the substrate. This ensures a more even distribution of reactive functional groups on the surface of the substrate. Following application of the silane layer, the silanated substrate may be baked to polymerize the silanes on the surface of the substrate and improve the reaction between the silane reagent and the substrate surface. Baking typically takes place at temperatures in the range of from 90° C. to 120° C., for example at 110° C., for a time period of from about 1 minute to about 120 minutes, for example for 60 minutes or about 60 minutes. In another embodiment, the time period is about 10 minutes, about 20 minutes, about 30 minutes, about 40 minutes, about 50 minutes, about 60 minutes, about 70 minutes, about 80 minutes, about 90 minutes, about 100 minutes, about 110 minutes or about 120 minutes.

In alternative aspects, as noted above, the silane functionalized compounds of the invention may be contacted with the surface of the substrate using controlled vapor deposition methods or spray methods. These methods involve the volatilization or atomization of the silane solution into a gas phase or spray, followed by deposition of the gas phase or spray upon the surface of the substrate, usually by ambient exposure of the surface of the substrate to the gas phase or spray. Vapor deposition typically results in a more even application of the derivatization solution than simply immersing the substrate into the solution.

The efficacy of the derivatization process, e.g., the density and uniformity of functional groups on the substrate surface, may generally be assessed by adding a fluorophore which binds the reactive groups, e.g., a fluorescent phosphoramidite such as Fluoreprime™ from Pharmacia, Corp., Fluoredite™ from Millipore, Corp. or FAM™ (carboxyfluorescine such as 5-carboxyfluorescine, 6-carboxyfluorescine or mixtures of 5- and 6-carboxyfluorescine) from ABI, and looking at the relative fluorescence across the surface of the substrate.

In one embodiment, novel processes are disclosed for forming an array of polymers by functionalizing a surface that includes covalently attaching a functionalized silicon compound. Modified hydrazone surfaces are produced by two methods: (1) reacting surfaces with aldehyde and oligonucleotide with hydrazine compounds and (2) reacting surfaces with hydrazine and oligonucleotide with aldehyde compounds.

In one embodiment, a plurality of the silane functionalized compounds are covalently attached to the surface of a solid substrate to provide a coating comprising derivatizable functional groups on the substrate, thus permitting arrays of immobilized oligomers to be covalently attached to the substrate via covalent reaction with the derivatizable functional groups. The immobilized oligomers, such as polypeptides, nucleic acids (polynucleotides) or analogs thereof, can be used in a variety of binding assays including biological binding assays. In another embodiment, high density arrays of immobilized nucleic acid probes may be formed on the substrate, and then one or more target nucleic acids comprising different target sequences may be screened for binding to the high density array of nucleic acid probes comprising a diversity of different potentially complementary probe sequences. For example, methods for light-directed synthesis of DNA arrays on glass substrates is described in McGall et al., J. Am. Chem. Soc., 119: 5081-5090 (1997), the disclosure of which is incorporated herein.

Detailed description of various silanes, for example, (HBAPTES) N-(2-hydroxyethyl)-N,N-bis-(trimethoxysilylpropyl)amine can be found in U.S. Pat. Nos. 6,262,216, 6,486,287, 6,429,275, 6,410,675, 6,743,882, 7,129,307, 7,125,947, 7,098,286, and 7,129,308, which are herein incorporated by reference in their entirety for all purposes.

Surface Functionalization

Methods are disclosed herein for immobilizing oligonucleotides on functionalized surfaces, for example functionalized microparticles. In some embodiments, reactive functionalities are introduced to both the surface of the substrate, for example, SiO₂, and to the oligonucleotides that are to be attached to the surface. The chemistry used for linking oligonucleotides to a glass support can be specific, fast and can provide stable chemical bonds. Stability of the chemically “activated” particles, substrates and oligonucleotides is also important for good shelf-life, and reproducibility.

Methods of functionalizing a surface to create modified hydrazone surfaces are provided according to one aspect. The methods include introducing surface hydrazine or surface aldehyde groups and then reacting with oligonucleotides functionalized with aldehydes or hydrazines. In one embodiment, utilization of an aldehyde-modified substrate is in combination with oligonucleotides containing a more strongly nucleophilic modification such as an alkyl or aryl hydrazine, hydrazide, or semicarbazide. This reaction forms a hydrazine linkage, and is chemospecific and rapid at pH 5-7, since hydrazines are more nucleophilic, yet less basic than alkylamines. The resulting hydrazone is analogous to a Schiff's base, but considerably more resistant to hydrolysis. Therefore, while hydrazones can be reduced with cyanoborohydride to increase the linkage stability, this can be an optional, and in some cases an unnecessary step. See Zatsepin et al., Bioconjugate Chem. 2005, 16:471, which is hereby incorporated by reference in its entirety.

In another embodiment, a surface with one or more hydrazine moieties is employed. In a further embodiment, one or more oligonucleotide compounds are attached to the surface through an aldehyde function, present on the one or more oligonucleotide compounds (see Formulae 9, 10 and 10(A), herein). Hydrazone stability increases in the following order: aromatic hydrazine (Ar—NHNH₂)>aliphatic hydrazine (R—NHNH₂)>hydrazide (CO—NHNH₂). See Sayer, et al. J. Amer. Chem. Soc. 1973, 95:4277, which is hereby incorporated by reference in its entirety for all purposes.

Similarly, aromatic aldehydes form more stable hydrazones than alkyl aldehydes. See Kale, et al., Bioconj. Chem. 2007, 18: 363-370, which is hereby incorporated by reference in its entirety for all purposes. In one embodiment, the combination of aromatic-hydrazine and aromatic-aldehyde is used if one intends to maximize stability of the resulting hydrazone, and potentially eliminate the need for borohydride reduction to stabilize the hydrazone linkage.

A variety of reagents are available for introduction of aromatic aldehyde and hydrazines directly onto the surface of glass substrates for the derivitizaton of encoded microparticles with oligonucleotides. The chemistry provided herein, used for linking oligonucleotides to a glass support can be specific, fast and provides stable chemical bonds. Stability of the chemically activated substrates (e.g., particles) and oligonucleotides is also important for good shelf-life, and reproducibility.

Most immobilization chemistries involve reaction of an electrophilic glass surface with a nucleophilic oligonucleotide modification. DNA synthesis reagents for preparing alkylamine-modified oligonucleotides are commercially available and provide a convenient nucleophilic linker. Electrophilic surface functional groups such as epoxide, isocyanate, isothiocyanate, N-hydroxysuccinimide ester, etc. have been introduced via silanation, but the chemical instability of these reactive groups, especially under the conditions in which the amine is optimally reactive (high pH), can result in variation in performance and limited storage shelf-life. Glass supports coated with milder electrophilic functional groups such as aldehydes or carboxylic acids are also available via direct silanation procedures.

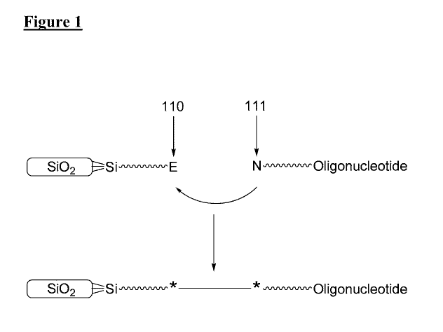

FIG. 1 illustrates a reaction of an electrophilic (“E”, 110) glass surface with a nucleophilic (“N”, 111) oligonucleotide modification. In one embodiment, the nucleophilic group is NH₂.

The functional groups (“E”, “N”) are easily introduced. In one embodiment, the functional groups are introduced in one step, for example, during the silanation or oligonucleotide synthesis process. In another embodiment, the functional groups are from readily available and inexpensive reagents and are thermally and hydrolytically stable. The coupling reaction, in one embodiment, is both rapid and efficient. In one embodiment, the coupling reaction is chemo-selective, for example, coupling is greater than hydrolysis. In a further embodiment, the coupling reaction provides stable chemical bonds (e.g., non-reversible) and is reproducible.

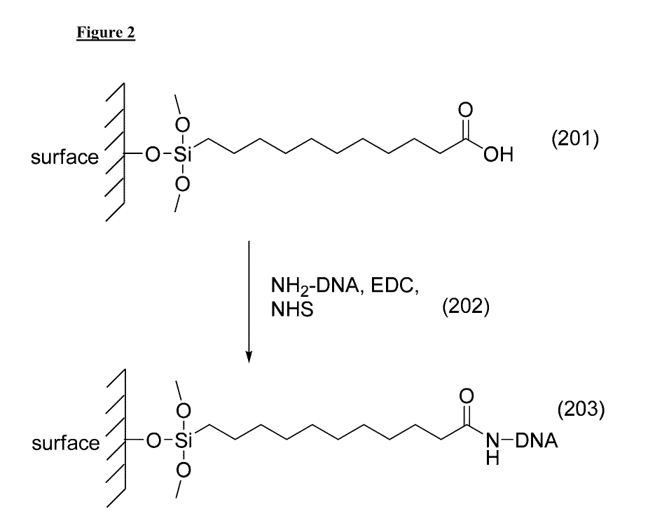

FIG. 2 illustrates a reaction of a glass support coated with milder electrophilic functional groups. Glass supports coated with milder electrophilic functional groups such as aldehydes or carboxylic acids are also available via direct silanation procedures. Although carboxylate supports are stable, coupling alkylamine-modified oligonucleotides to them requires the use of activating agents, such as carbodiimides (e.g., EDC, DSC, CDI, etc.) to generate reactive carbonyl species which are unstable, again leading to potential variability.

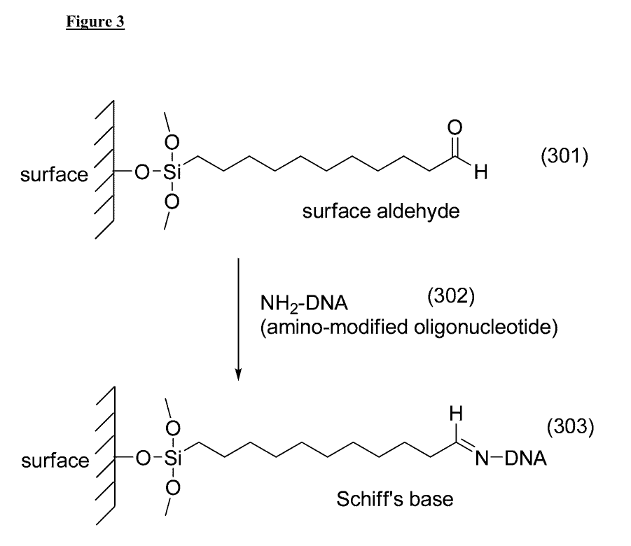

FIG. 3 illustrates a reaction of a surface aldehyde with an alkylamine-modified oligonucleotide. Aldehyde-modified supports are also stable, and available by direct silanation. Aldehydes also react covalently with alkylamine-modified oligonucleotides, but they do so rather sluggishly, and the resulting Schiffs base linkage is unstable to hydrolysis and must be reduced with sodium cyanoborohydride (NaCNBH₃) to provide adequate stabilization.

Surveys of immobilization chemistries for oligonucleotide beads and microarrays have appeared in several publications, for example:

Hermanson G T, Bioconjugate Techniques, 2nd Edition. Elsevier, 2008.

Heise C, Bier F F. Immobilization of DNA on Microarrays. In: Immobilization of DNA on Chips II, Topics in Current Chemistry, Wittmann C, Ed. 2005, 261: 1-25. Springer Berlin/Heidelberg.

Luderer F, Walschus U, Immobilization of Oligonucleotides for Biochemical Sensing by Self-Assembled Monolayers: Thiol-Organic Bonding on Gold and Silanization on Silica Surfaces. In: Immobilisation of DNA on Chips I. Topics in Current Chemistry, Wittmann C, Ed. 2005, 260: 77-111. Springer Berlin/Heidelberg

Steinberg G, et al., Strategies for Covalent Attachment of DNA to Beads, Biopolymers 2004, 73: 597-605.

Pirrung M, How to Make a DNA Chip, Angew. Chem. Int. Ed. 2002, 41: 1276-1289;

Lindroos K, et al., Minisequencing on Oligonucleotide Microarrays: Comparison of Immobilisation Chemistries, Nucleic Acids Res., 2001, 29: 69;

Beaucage S L, Strategies in the preparation of DNA Oligonucleotide Arrays for Diagnostic Applications, Current Med. Chem. 2001, 10: 1213-44;

Zammatteo N, et al., Comparison between Different Strategies of Covalent Attachment of DNA to Glass Surfaces to Build DNA Microarrays, Anal. Biochem. 2000, 280: 143-150, each of which is hereby incorporated by reference in its entirety.

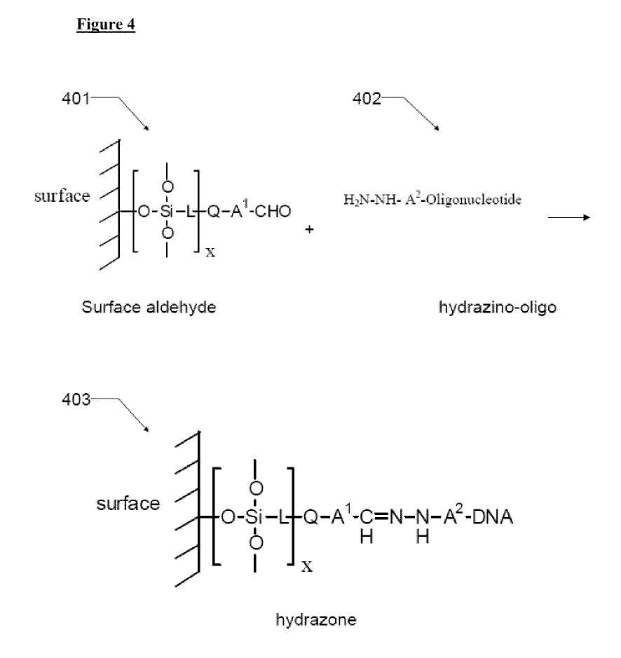

A reaction of a surface aldehyde with a hydrazino-modified oligonucleotide is illustrated in FIG. 4. See 7,129,229 Raddatz et al., “Hydrazide Building Blocks and Hyrazide Modified Biomolecules,” the disclosure of which is incorporated herein.

In one embodiment, a method of functionalizing a surface is provided. The method comprises covalently attaching to the surface a plurality of functionalized silicon compounds wherein each of the plurality of functionalized silicon compounds comprises at least one derivatizable functional group and at least one activated silicon groups. In a further embodiment, at least one of the functionalized silicon compounds comprises a plurality of activated silicon groups, for example, 2, 3, 4 or more activated silicon groups. The method may further comprise forming an array of nucleic acids by covalently attaching a plurality of nucleic acids to the surface through the functionalized silicon compounds.

### A. Dipodal Hydrazone Formed by Introducing an Aldehyde Group.

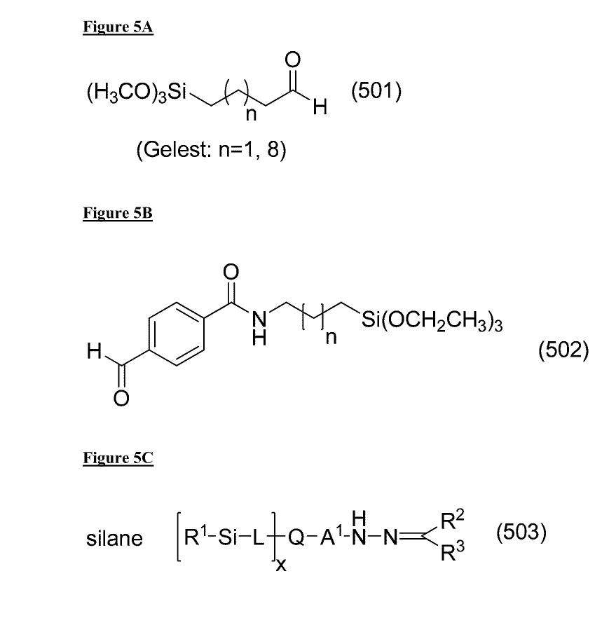

In one aspect, a surface with an aldehyde moiety is employed, and the hydrazino group is attached to the oligonucleotide. FIG. 4 illustrates a reaction of a surface aldehyde with a hydrazino-modified oligonucleotide. Reagents for introducing aldehyde groups using Formula 1 are illustrated in FIGS. 5A and 5B. The aldehyde silanes depicted in structure 5A are commercially available from Gelest, Inc. (Morrisville, Pa.). The p-carboxybenzaldehyde silane 5B has been described by Tsubuku, et al. (PCT Int. Appl. (2009), WO 2009044697).

### Dipodal Silanes

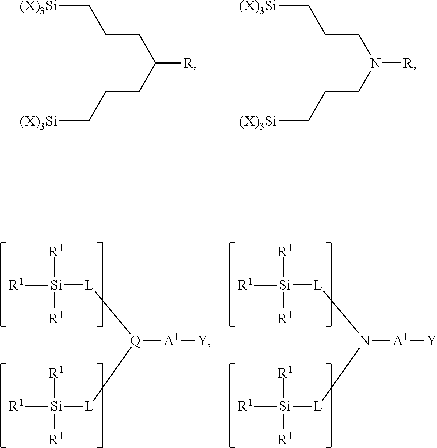

See Formula 1 for definition of variables R¹, L, Q, A¹ and Y.

Functional dipodal silanes and combinations of non-functional dipodal silanes with functional conventional silanes have a significant hydrolytic stability to substrate bonding. The fundamental step by which silanes provide adhesion is forming a —Si—O—Si bond with a glass substrate. The bond strength is defined by the bond dissociation energy of Si—O—Si and according to the equilibrium equation the bond dissociation K_{d} is ˜10⁻² for a single bond and, therefore, is increased to ˜10⁻⁶ for dipodal silanes of the type above. Detailed description of various silanes, for example, (HBAPTES) N-(2-hydroxyethyl)-N,N Bis (trimethoxysilyl propyl)amine can be found in U.S. Pat. Nos. 6,262,216, 6,486,287, 6,429,275, 6,410,675, 6,743,882, 7,129,307, 7,125,947, 7,098,286, and 7,129,308, which are hereby incorporated by reference in its entirety for all purposes.

### B. General Hydrazone to Introduce a Surface Aldehyde.

FIG. 5C illustrates a dipodal hydrazone according to one embodiment.

According to an embodiment, a method of functionalizing a surface is provided. The method comprises covalently attaching a functionalized silicon compound (which in some embodiments, is dipodal, e.g., x is 2), of Formula 1 or a plurality of functionalized silicon compounds of Formula 1 to a substrate.

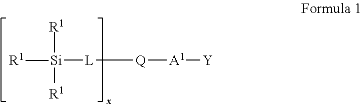

wherein, x is an integer selected from 1 to 3,

each occurrence of R¹ is independently any alkoxy, aryloxy or halogen or is a lower alkyl where at least 1 of the R¹ groups is an alkoxy or halogen,

each occurrence of L is independently a spacer group optionally comprising one or more organofunctional moieties comprising a functional group selected from the group consisting of ether, amine, sulfide, sulfoxyl, carbonyl, thione, ester, thioester, carbonate, thiocarbonate, carbamate, thiocarbamate, amide, thioamide, urea and thiourea group,

Q is N, C₁-C₁₀ alkyl or C₁-C₁₀ substituted alkyl,

A¹ is a linking group comprising a straight chain alkyl, branched alkyl, cycloalkyl, alkenyl, alkynyl, aryl or heteroaryl, optionally comprising one or more organofunctional moieties selected from ether, amine, sulfide, sulfonyl, sulfate, carbonyl, thione, ester, thioester, carbonate, thiocarbonate, carbamate, thiocarbamate, amide, thioamide, urea and thiourea group, and

Y is a derivatizable functional group or protected functional group selected from the group consisting of halogen, hydroxy, thiol, amine, hydrazine, aminooxy, sulfonate, sulfate, azide, carbonyl, carboxyl, carboxylate, thiocarboxyl, aldehyde, alkene, alkyne, disulfide, isocyanate, isothiocyanate or modified forms thereof.

In one embodiment of Formula 1, a dipodal silicon compound or tripodal silicon compound is provided (e.g., x is 2 or 3), shown in Formula 1(A) and 1(B), respectively.

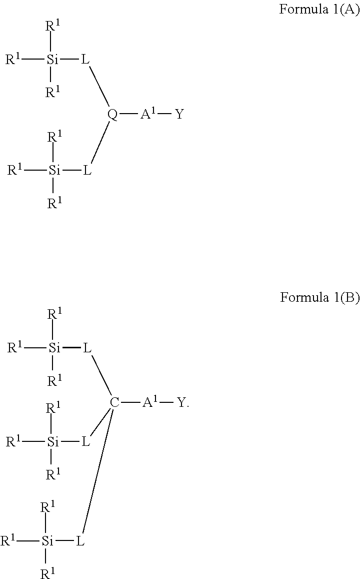

For Formulae 1(A) and 1(B), R¹, L, Q, A¹ and Y are defined as provided for Formula 1.

In one embodiment of compounds of Formula 1, x is 2 and Q is N. In another embodiment of compounds of Formula 1, x is 2 and Q is —CH₂—. In yet another embodiment of compounds of Formula 1, x is 3 and Q is C₁-C₁₀ alkyl or C₁-C₁₀ substituted alkyl. Q is methyl, ethyl, propyl or —N— in one Formula 1(A) embodiment.

In one embodiment of the compounds of Formula 1, if x is 3, then Q is methyl. In one embodiment, at least one occurrence of L is an aliphatic chain comprising at least two carbon atoms.

In one Formula 1(A) embodiment, A¹ is a straight chain alkyl. In a further Formula 1(A) embodiment, A¹ comprises one or more organofunctional moieties. In yet a further Formula 1(A) embodiment, A¹ is a C₃, C₄, C₅, C₆, C₇, C₈, C₉ or C₁₀ straight chain alkyl. In even a further Formula 1(A) embodiment, A¹ comprises a carboxyl group.

In one embodiment of Formula I(A), each L group is a carbon chain having 3, 4, or 5 carbon atoms, Q is N, and A¹ and Y together, form the group

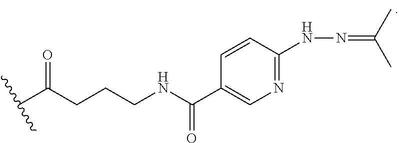

For example, in one embodiment, A¹ is —C(═O)CH₂CH₂NHC(═O)— and Y is 2-(2-(propan-2-ylidene)hydrazinyl)pyridine. In one embodiment, this compound can be attached to a substrate, for example, a microparticle. In a further embodiment, a plurality of compounds are attached to one or more microparticles.

In another Formula I(A) embodiment, each L group is a carbon chain having 3, 4, or 5 carbon atoms, Q is N, and A¹ and Y together, form the group

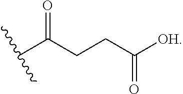

For example, in one embodiment, A¹ is a C₃ straight chain alkyl comprising a carboxyl moiety (e.g., —C(═O)CH₂CH₂—) and Y is COOH. In a further embodiment, each L group has 3 carbons. In one embodiment, this compound can be attached to a substrate, for example, a microparticle. In a further embodiment, a plurality of compounds are attached to one or more microparticles.

In another embodiment of compounds Formula 1, L and A¹ are independently selected from —(CH₂)ₙ—, —C(═O)—, —C(═O)CH₂CH₂—, —CH₂C(═O)—, —CH₂C(═O)NH—, —CH₂C(aromatic ring)NH—. In a further embodiment, when L or A¹ is —(CH₂)ₙ—, the carbon chain defined by n is 2, 3, 4 or 5 atoms long.

It is understood that the various chemical groups or moieties exemplified herein for L, Q, A¹, and Y and other groups in the Formulae disclosed herein can have any chemically reasonably valencies. For example, when x is 3 and Q is “methyl” in Formula 1(A), above, it is understood that Q has a single carbon atom and corresponds to:

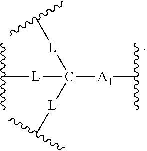

Likewise, when x is 3 and Q is “ethyl”, Q has two carbon atoms and includes:

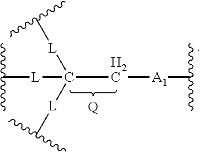

Similarly, when x is 2 and Q is ethyl, compounds can include:

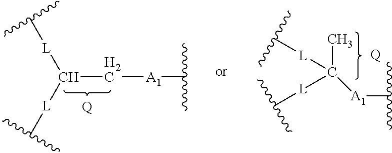

In one embodiment, a compound of Formula 1 can be attached to a surface, to form a modified surface of Formula 2:

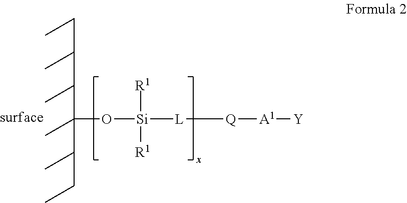

wherein, R¹, L, x, Q, A¹ and Y are defined as provided for Formula 1.

In one embodiment, a surface of Formula 2 has multiple R¹ moieties are covalently bound to the surface.

It is to be understood that when an organosilane is bound to a silica surface (e.g., a structure of Formula 2), one or more R¹ groups can be bound to the surface. Organosilanes form disordered films with a random network of Si—O—Si bonds. Therefore, the exact structure of the modified surface cannot be define precisely. For example, some of the Si—O—Si bonds are formed between adjacent organosilane moieties; some R¹ groups are left unreacted (see Formula 2), and some are bonded directly to the surface (see Formula 2).

Modified surfaces of Formulae 2 and 2(A) include structures in which at least one of the Si—O— groups is attached to the surface. The remaining two Si—O— groups can form one or more —Si—O—Si— linkages with adjacent silyl moieties derived from compounds of Formula 1. Alternatively or additionally, additional Si—O—Si linkages can be formed with the surface. For example, one of the Si—O— moieties are attached to the surface, and the remaining two Si—O— moieties are attached to adjacent silyl moieties, or two of the Si—O— moieties are attached to the surface and one of the remaining Si—O— moieties is attached to an adjacent silyl moiety, or all three Si—O— moieties are attached to the surface, or one moiety is attached to the surface and one or two of the remaining moieties are left unreacted. For example, after surface treatment with a compound of Formula 1 in which each of R¹ is an alkoxy, aryloxy or halogen, each silyl group is attached to the surface by one or more Si—O— linkage, and the remaining Si—O— linkages are attached to adjacent silyl groups by —Si—O—Si— linkages.

Accordingly, in one embodiment of surfaces of Formula 2, a structure of Formula 2(A) is provided (2 R¹ groups are left unreacted).

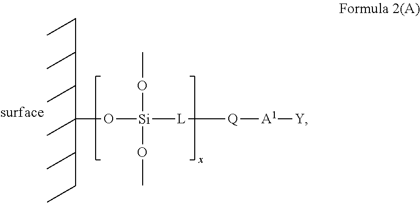

wherein, L, x, Q, A¹ and Y are defined as provided for Formula 1.

In one embodiment of surfaces of Formula 2, Y is selected from Cl, Br, I, mesylate, methyl sulfonic (OMs), OTs, OH, SH, NH₂, ONH₂, NHNH₂, COOH, COSH, N₃, CH═CH₂ and C═CH. In a further embodiment, Q is —N— or —CH₂—.

In another embodiment of surfaces of Formula 2, L and A¹ are independently selected from —(CH₂)ₙ—, —C(═O)—, —CH₂C(═O)—, —CH₂C(═O)NH—, —CH₂C(aromatic ring)NH—. In a further embodiment, when L or A¹ is —(CH₂)ₙ—, the carbon chain defined by n is 2, 3, 4 or 5 atoms long.

In one embodiment of Formula 2 or 2(A), Q is N or methyl. In a further embodiment, L is methyl, ethyl or propyl.

In another embodiment of structures of Formula 2 and 2(A), L and A¹ are independently selected from —(CH₂)ₙ—, —C(═O)—, —CH₂C(═O)—, —CH₂C(═O)NH—, —CH₂C(aromatic ring)NH—. In a further embodiment, when L or A¹ is —(CH₂)ₙ—, the carbon chain defined by n is 2, 3, 4 or 5 atoms long.

In yet another embodiment of structures of Formula 2, at least one of L and A¹ is selected from —C(═O)—, —CH₂C(═O)— and —CH₂C(═O)NH—.

Another embodiment of structures of Formula 2 includes compounds having a Y group having at least one of the following moieties: halogen, hydroxy, thiol, amine, hydrazine, aminooxy, sulfonate, sulfate, carbonyl, carboxyl, carboxylate, thiocarboxyl, aldehyde, alkene.

According to another embodiment, a method for functionalizing a surface is provided. The method comprises covalently attaching a plurality of derivatizable functionalized silicon compounds of Formula 1 to the surface, to form a modified surface of Formulae 2, 2(A), 2(B) or 2(C), and covalently attaching an array of nucleic acids (polynucleotides) to the modified surface through the derivatizable functionalized silicon compounds. The derivatizable functional group can be the same or different for each compound of Formula 1, and in one embodiment, is selected from the group consisting of halogen, hydroxy, thiol, amine, hydrazine, aminooxy, sulfonate, sulfate, azide, carbonyl, carboxyl, carboxylate, thiocarboxyl, aldehyde, alkene, alkyne, disulfide, isocyanate, isothiocyanate or modified forms thereof. In a further embodiment, the surface is the surface of an encoded microparticle.

The functionalized silicon compounds each include an activated silicon group and a derivatizable functional group. Exemplary derivatizable functional groups include hydroxyl, amino, carboxyl and thiol, as well as modified forms thereof, such as activated or protected form, for example, activated hydroxyl groups or protected hydroxyl groups. The functionalized silicon compounds may be covalently attached to surfaces to form functionalized surfaces which may be used in a wide range of different applications. In one embodiment, the silicon compounds are attached to the surface of a substrate comprising silica, such as a glass substrate, to provide a functionalized surface on the silica containing substrate, to which molecules, including polypeptides and nucleic acids, may be attached.

In one embodiment, after covalent attachment of a functionalized silicon compound to the surface of a solid silica substrate to form a functionalized coating on the substrate, an array of nucleic acids may be covalently attached to the substrate through the functionalized coating. Thus, the method permits the formation of high density arrays of nucleic acids immobilized on a substrate, which may be used in conducting high volume nucleic acid hybridization assays.

According to one embodiment, a method for functionalizing a surface is provided. The method entails covalently attaching a functionalized silicon compound of Formula 3 to a surface.

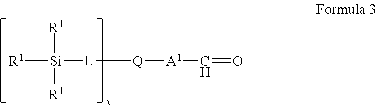

wherein, x is an integer selected from 1 to 3,

each occurrence of R¹ is independently any alkoxy, aryloxy or halogen, or is a lower alkyl where at least 1 of the R¹ groups is an alkoxy or halogen,

each occurrence of L is independently a spacer group optionally comprising one or more organofunctional moieties comprising functional groups selected from the group consisting of ether, amine, sulfide, sulfoxyl, carbonyl, thione, ester, thioester, carbonate, thiocarbonate, carbamate, thiocarbamate, amide, thioamide, urea and thiourea groups,

Q is N, C₁-C₁₀ alkyl or C₁-C₁₀ substituted alkyl and,

A¹ is a linking group comprising straight chain or branched alkyl, cycloalkyl, alkenyl, alkynyl, aryl or heteroaryl; optionally comprising one or more organofunctional moieties selected from the group consisting of ether, amine, sulfide, sulfonyl, sulfate, carbonyl, thione, ester or thioester, carbonate or thiocarbonate, carbamate or thiocarbamate, amide or thioamide, urea and thiourea groups.

In one embodiment, x is 2. In a further embodiment, Q is N or methyl. In one embodiment, if x is 3, then Q is methyl or ethyl.

In one embodiment, one occurrence of L is an aliphatic chain comprising at least two atoms. In a further embodiment, one occurrence of L is an aliphatic chain comprising two atoms.

In one embodiment, the covalent attachement of a compound of Formula 3 to a surface results in the aldehyde modified surface of Formula 4, 4(A) or 4(B). R¹ can be any of the groups specified above. In the case of Formulae 4(A) and 4(B), x is 2. As stated above, Formulae 4, 4(A), 4(B) and 4(c) are meant to be exemplary, as the organosilane network formed between the compounds and the silicon surface cannot be defined precisely.

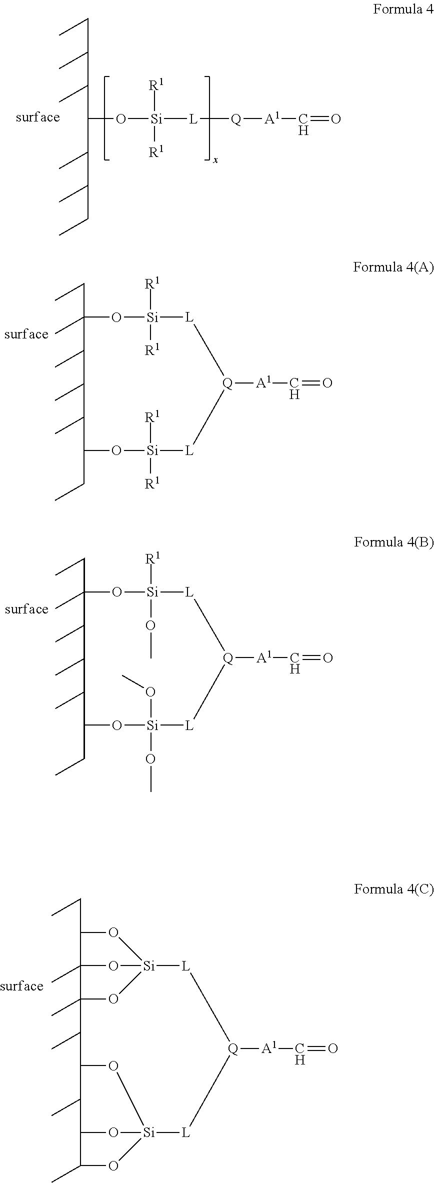

wherein, R¹, L, x, Q and A¹ are defined as provided for Formula 3.

In one Formula 4 embodiment, a surface of Formula 4(D) is provided.

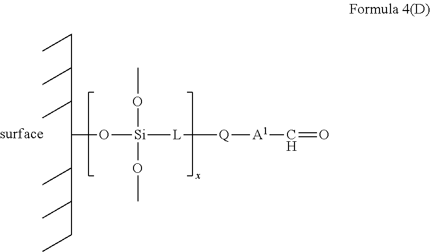

wherein, L, x, Q and A¹ are defined as provided for Formula 3.

In one embodiment, a compound of Formula 5, or a plurality of compounds of Formula 5, are reacted with the surface of Formula 4 or 4(A) to produce the oligonucleotide derivatized surface of Formula 6.

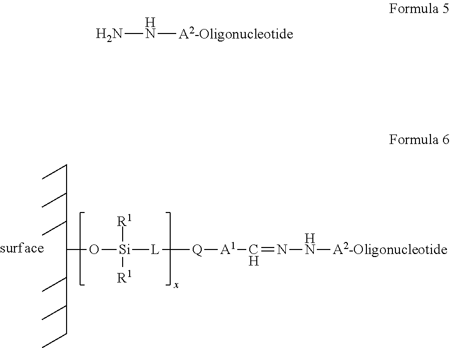

wherein, R¹, L, x, Q and A¹ are defined as provided for Formula 3, and

A² is a linking group comprising straight chain or branched alkyl, cycloalkyl, alkenyl, alkynyl, aryl or heteroaryl; optionally comprising one or more organofunctional moieties comprising a functional group selected from the group consisting of ether, amine, sulfide, sulfonyl, sulfate, carbonyl, thione, ester or thioester, carbonate, thiocarbonate, carbamate, thiocarbamate, amide, thioamide, urea and thiourea.

In one Formula 6 embodiment, each R¹ is methoxy, as provided below for Formula 6(B).

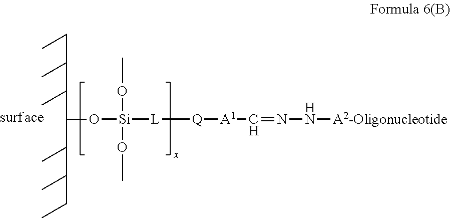

In one embodiment of Formula 6, x is 2. In a further embodiment, A¹ and A² are the same.

In one embodiment of Formulae 6, Q is N or methyl. In a further embodiment, A¹ is methyl, ethyl or propyl.

In another embodiment of structure of Formula 6, each A¹ is independently selected from —(CH₂)ₙ—, —C(═O)—, —CH₂C(═O)—, —CH₂C(═O)NH—, —CH₂C(aromatic ring)NH—. In a further embodiment, when A¹ is —(CH₂)ₙ—, the carbon chain defined by n is 2, 3, 4 or 5 atoms long.

In yet another embodiment of structures of Formulae 6, at least one of L and A¹ is selected from —C(═O)—, —CH₂C(═O)— and —CH₂C(═O)NH—.

### C. Specific Aldehyde-Modified Silane Compound to Introduce a Surface Aldehyde.

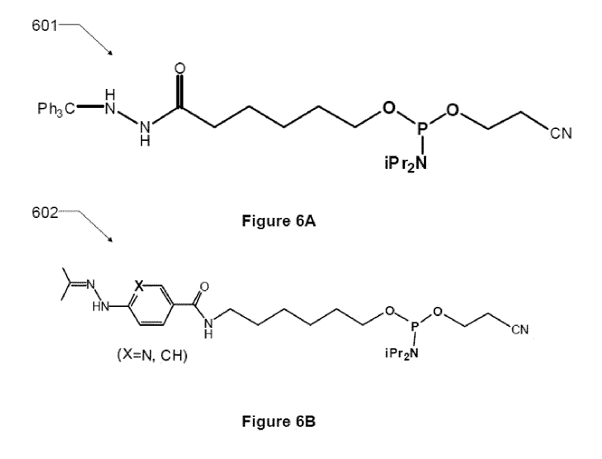

Reagents (compounds 601 and 602) for introducing hydrazine groups on the oligonucleotide are illustrated in FIGS. 6A and 6B. See also Raddatz, et al. N.A.R. 2002, 30:4793). In one embodiment, compound 601 is used to introduce hydrazine onto an oligonucleotide. In a further embodiment, the oligonucleotide is covalently attached to a silanated surface. In another embodiment, compound 602 is used to introduce hydrazine onto an oligonucleotide. In a further embodiment, the oligonucleotide is covalently attached to a silanated surface.

### D. Dipodal Hydrazone Formed by Introducing a Dipodal Hydrazine Group.

According to another embodiment, a surface with at least one hydrazine moiety is employed, and an oligonucleotide compound with an aldehyde group is attached to the surface through the hydrazine moiety. In a further embodiment, a plurality of oligonucleotide compounds, each with an aldehyde group, is attached to the surface through a plurality of hydrazine moieties.

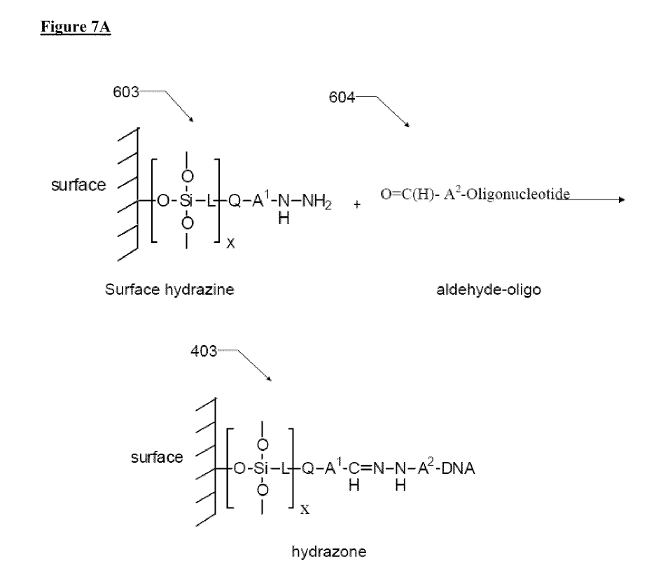

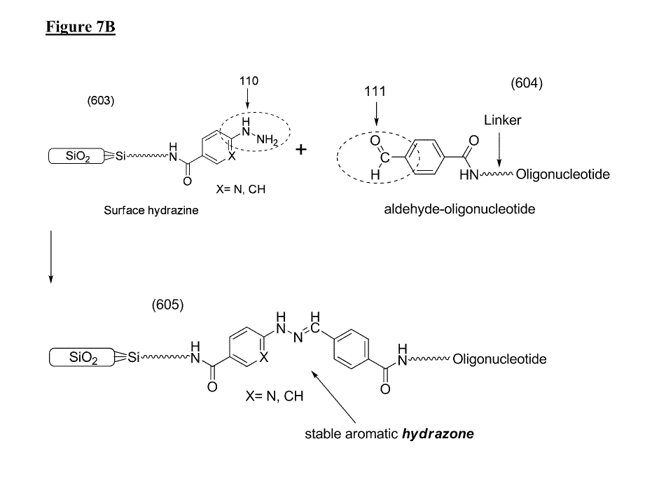

FIGS. 7A and 7B illustrate a reaction of a surface hydrazine with an aldehyde-modified oligonucleotide according to one embodiment. In this example, hydrazine is the nucleophilic group (E, 110) and the aldehyde is the electrophilic group (N, 111) as shown in FIG. 1. See U.S. Pat. No. 5,420,285, Schwartz et al., “Protein Labelling Utilizing Certain Pyridyl Hydrazines, Hydrazides and Derivatives,” the disclosure of which is incorporated herein.

Hydrazone stability increases in the order: aromatic hydrazine (Ar—NHNH₂)>aliphatic hydrazine (R—NHNH₂)>hydrazide (CO—NHNH₂). See Sayer, et al. J. Amer. Chem. Soc. 1973, 95:4277, the disclosure of which is incorporated herein. Similarly, aromatic aldehydes form more stable hydrazones than alkyl aldehydes. See Kale et al., Bioconj. Chem. 2007, 18: 363-370, the disclosure of which is incorporated herein. In one embodiment, the combination of aromatic-hydrazine and aromatic-aldehyde is used if one intends to maximize stability of the resulting hydrazone, and potentially obviate the need for borohydride reduction to stabilize the hydrazone linkage.

An exemplary compound of a surface hydrazine silicon compound is shown below

### F. General Hydrazone to Introduce a Surface Hydrazine.

According to one embodiment, a method is provided for functionalizing a surface. The method comprises covalently attaching a functionalized silicon compound of Formula 7 to a surface, to form to structure of Formula 8.

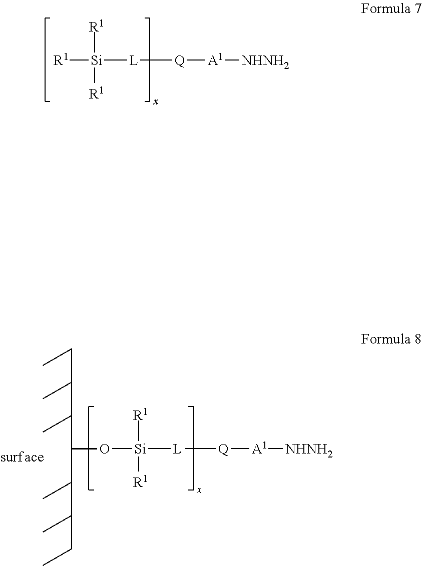

wherein, R¹, L, X, Q and A¹ are defined as provided for Formula 3.

In one embodiment of Formula 7 or 8, x is 2. In another embodiment of Formula 7 or 8, if x is 3, then Q is methyl or ethyl.

In one embodiment of Formula 8, Q is N. In a further embodiment, L is methyl, ethyl or propyl. In another embodiment of surfaces of Formula 8, Q is methyl, ethyl or propyl.

In one particular embodiment, a hydrazine modified surface structure of Formula 8(A) is provided:

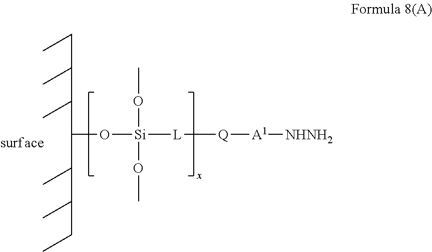

wherein, L, X, Q and A¹ are defined as provided for Formula 8.

In one embodiment of Formula 8(A), Q is N and x is 2. In another embodiment of Formula 8(A), Q is methyl and x is 2 or 3.

In another embodiment, surface structures of Formulae 10 and 10(A) are provided. A structure of Formula 10 can be formed by reacting the surface structure of Formula 8 with an oligonucleotide compound of Formula 9. A structure of Formula 10(A) can be formed by reacting the surface structure of Formula 8(A) with an oligonucleotide compound of Formula 9.

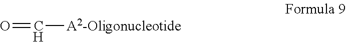

(See also compound 603, FIG. 7B):

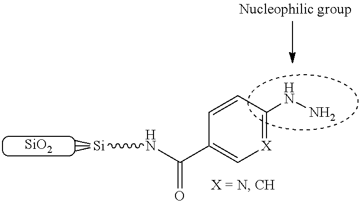

An exemplary compound of an aldehyde-oligonucleotide compound is shown below

(See also compound 604, FIG. 7B):

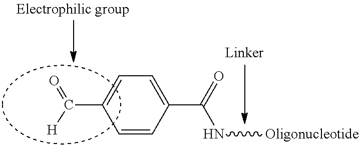

An exemplary compound of a hydrazone compound is shown below (See also compound 605, FIG. 7B):

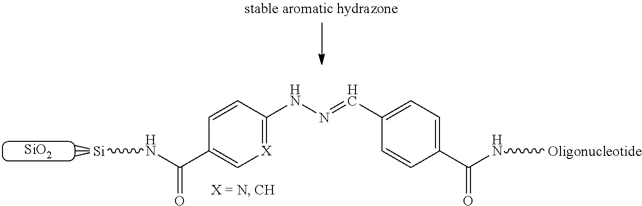

### E. Reagents for Introducing the Hydrazine on the Surface and Aldehyde on the Oligonucleotide.

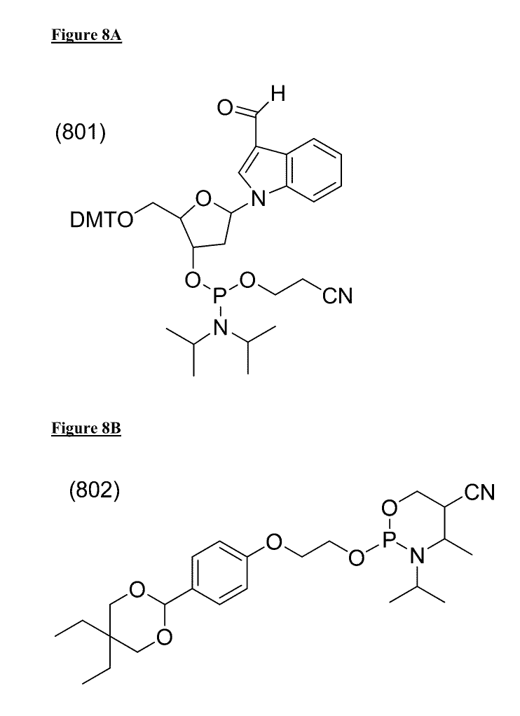

Reagents (compound 801 and 802) for introducing aldehyde groups into oligonucleotides are illustrated in FIGS. 8A and 8B, which are commercially available from Glen Research (Sterling, Va.). In one embodiment, compound 801 or 802 is used to introduce an aldehyde group onto an oligonucleotide to form a modified oligonucleotide compound.

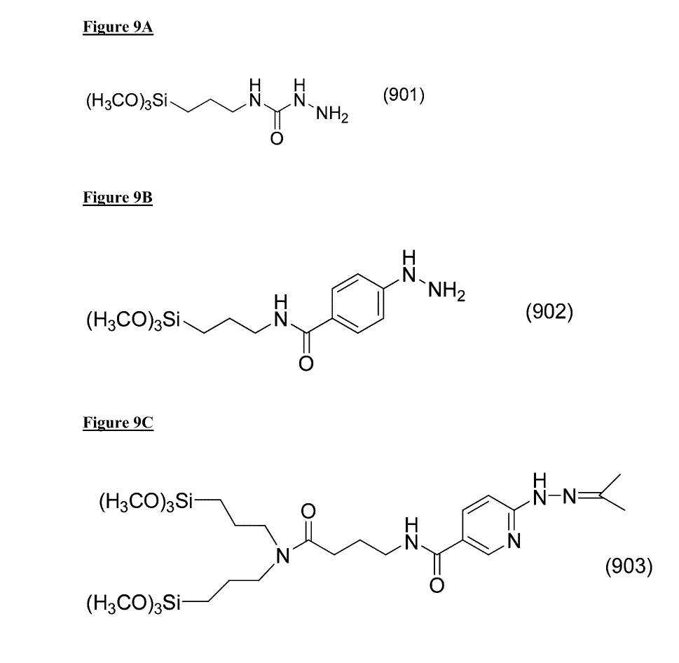

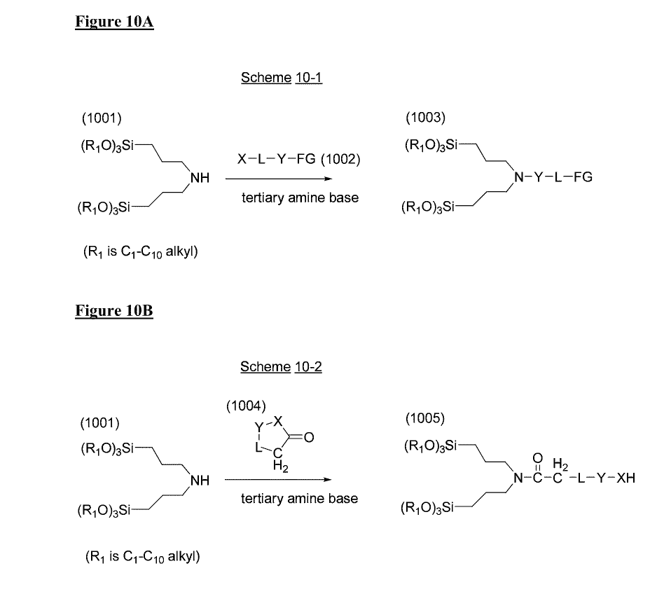

FIG. 9 illustrates reagents for introducing hydrazine groups to silica and other oxide surfaces. The reagents illustrated in FIGS. 9A and 9B are commercially available from Gelest, Inc. (Tullytown, Pa.) and from Solulink, Inc. (San Diego, Calif.) respectively.

In one embodiment, one or both of the reagents illustrated in FIG. 9 are used to modify a surface and at least one modified oligonucleotide compound is attached to the surface through the hydrazine group on the reagent.

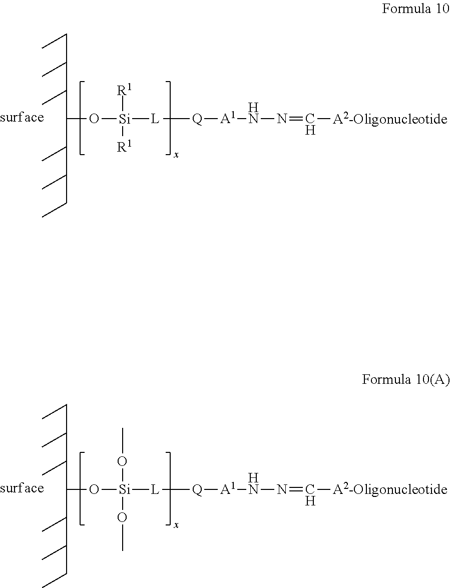

wherein, R¹, L, x, Q and A¹ and A² are defined as provided for Formulae 5 and 6.

In one embodiment of Formula 10, x is 2. In a further embodiment, Q is N or methyl.

In one embodiment of Formula 10 or 10(A), Q is N or methyl.

In another embodiment of Formula 10 or 10(A), if x is 3, then Q is methyl or ethyl.

According to another embodiment, a method of functionalizing a surface comprises covalently attaching a functionalized silicon compound of Formula 11 to a surface.

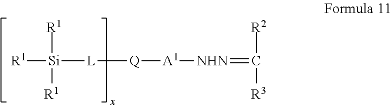

wherein, R¹, L, x, Q and A¹ are defined as provided for Formula 3 and R² and R³ are independently selected from H, alkyl, substituted alkyl, cycloalkyl and substituted cycloalkyl.

In one embodiment, a compound of Formula 11 is attached to a surface to form a structure represented by Formula 8, above. In a further embodiment, a structure of Formula 8(A) is provided by reacting a compound of Formula 11 with a surface.

In another embodiment, a surface structure of Formulae 10 or 10(A) is provided by reacting a compound of Formula 9 with a surface modified with at least one compound of Formula 11.

G. Specific hydrazine-modified surfaces utilizing dipodal silane to introduce a surface hydrazine.

According to one embodiment, a method of functionalizing a surface comprises covalently attaching a functionalized silicon compound to the surface, wherein the surface hydrazine structure includes the following compound of Formula 11 attached to a surface:

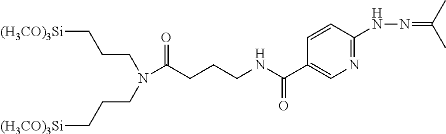

(x=2; Q is —N—, each occurrence of R¹ is methoxy; each occurrence of L is —(CH₂)₃—; A¹ is

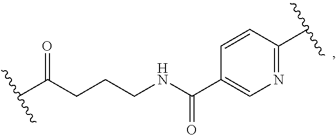

### and R² and R³ are CH₃.

FIG. 9C illustrates the above reagent for producing a Dipodal hydrazone (903) according to one embodiment.

### H. General Schemes 10-1 and 10-2 Utilizing the Dipodal Structure.

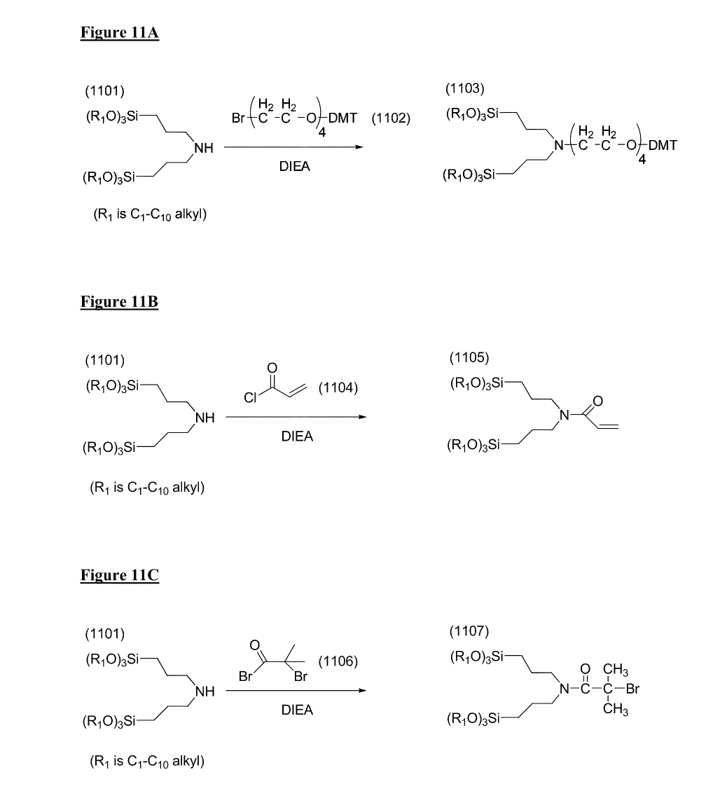

FIGS. 10A and 10B illustrate general schemes 10-1 and 10-2 according to an embodiment. In one general synthetic process, compounds of Formula 1 are prepared according to the reaction scheme as shown in FIG. 10A.

According to scheme 10-1 in FIG. 10A, an X—Y-L-FG composition is reacted with compound 1001 to prepare compound 1003.

X=leaving group (e.g., Cl, Br, I, mesylate or methyl sulfonic (OMs), p-toluene sulfonic or tosylate (OTs), etc.)

Y=first linking group (e.g., —(CH₂)ₙ—, —C(═O)—, —CH₂C(═O)—, —CH₂C(═O)NH—, —CH₂C(aromatic ring)NH—, etc.)

L=optional second linking group (e.g., (CH₂)ₙ, (OCH₂CH₂)ₙ; n is an integer from 0-20. In a further embodiment, n is an integer selected from 2, 3, 4, 5, 6, 7, 8, 9, 10, 11 or 12. In even a further embodiment, n is an integer selected from 2, 3, 4, 5 and 6.

FG=functional group or protected functional group (Cl, Br, I, (mesylate or methyl sulfonic (OMs), OTs, OH, SH, NH₂, ONH₂, NHNH₂, COOH, COSH, N₃, CH═CH₂, C═CH, etc.)

According to scheme 10-2 in FIG. 10B, a cyclic tertiary amine base composition is reacted with compound 1001 to prepare compound 1005.

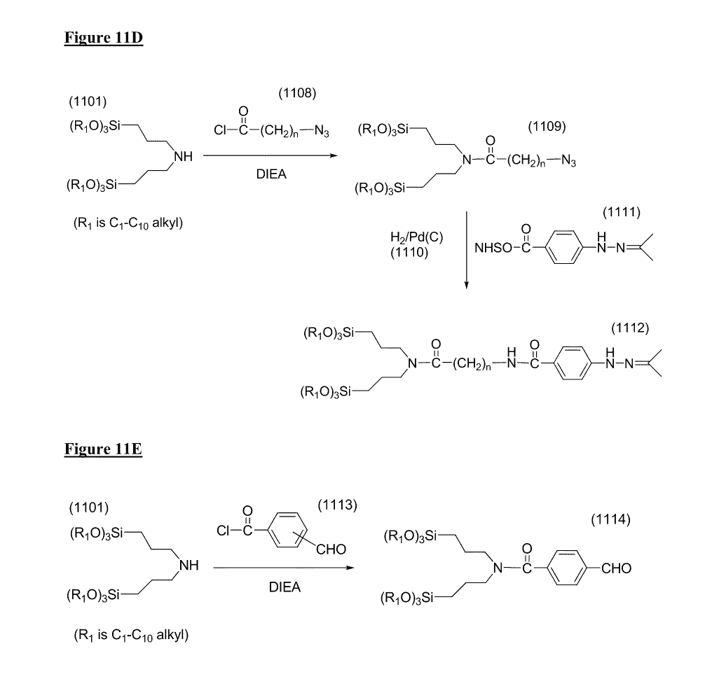

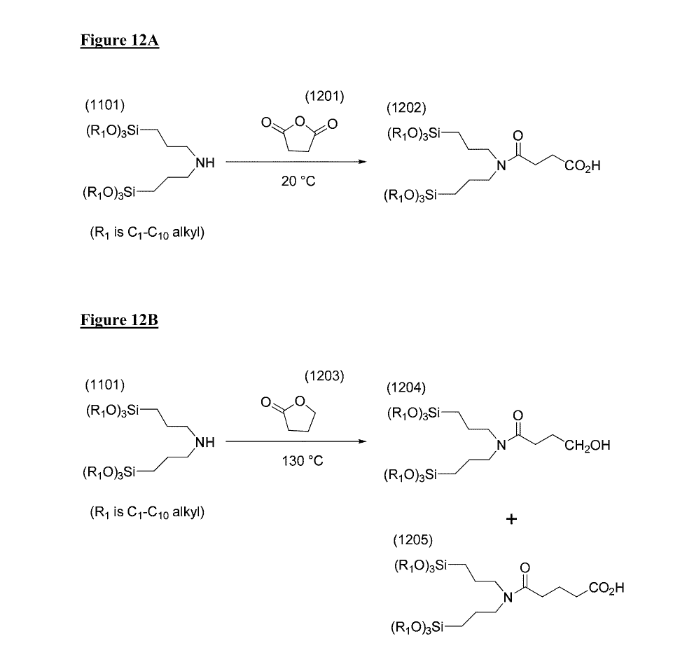

FIGS. 11A to 11E illustrate examples of reactions of the Formula illustrated in FIG. 10A according to an embodiment.

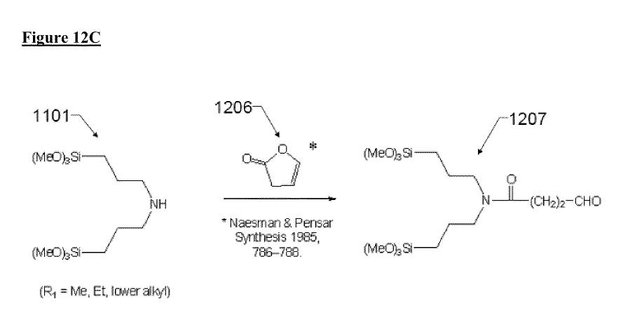

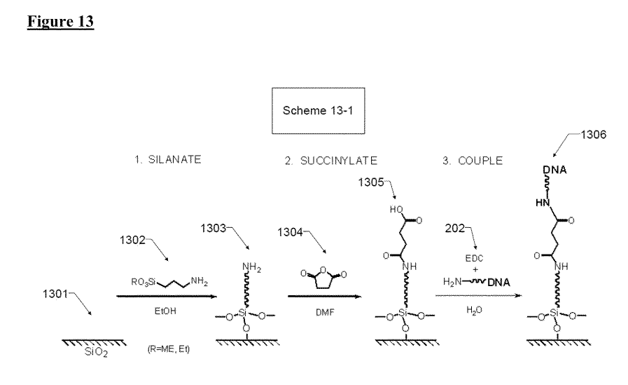

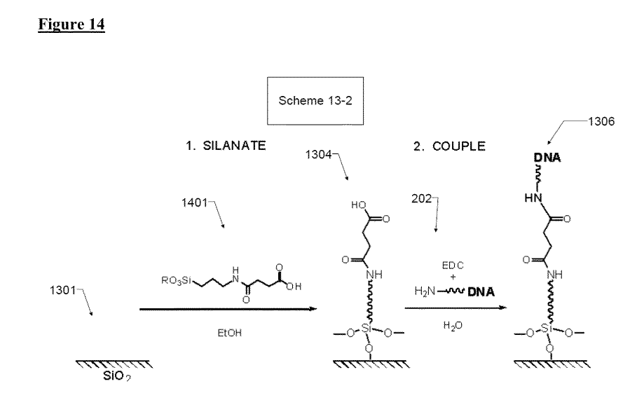

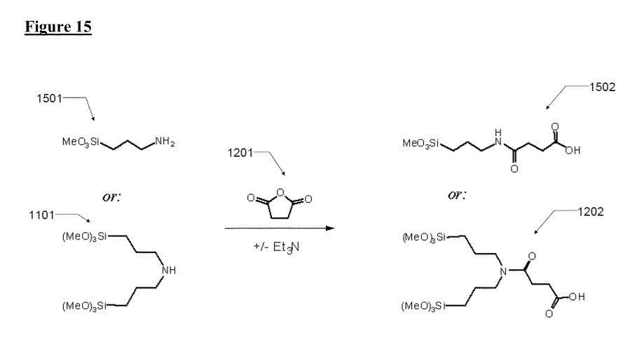

FIGS. 12A to 12C illustrate examples of reactions of the Formula illustrated in FIG. 10B according to an embodiment. The 3-H-Furan-2-one structure is further described in Naesman & Pensar Synthesis 1985, 786-788, which is hereby incorporated by reference in its entirety for all purposes.

### I. One Step Silanation Method Using Carboxylated Silanes.

One of the challenges for commercializing the Encoded Microparticles technology is to implement a rapid, reproducible and cost-efficient process for immobilizing oligonucleotides on the barcode particles. The chemistry used for linking oligonucleotides to a glass support must be specific, fast and provide stable chemical bonds. Large-scale, high-throughput manufacturing requires that the reagents and procedures that are employed to introduce reactive functionalities to both substrate (SiO₂) and oligonucleotides be simple and reproducible. Stability of the “activated” particles and oligonucleotides is another important requirement for good shelf-life, and reproducibility.

One current immobilization process involves reaction of a carboxylated glass surface with an amine-modified oligonucleotide in the presence of the carbodiimide activating agent 1-ethyl-3-dimethylaminopropyl carbodiimide (EDC). In this process, surface carboxyl groups are introduced in two steps: silanation with 3-aminopropyl trimethoxysilane (APTMS) followed by succinylation of the resulting aminated surface with succinic anhydride (See FIG. 13). FIG. 13 illustrates a scheme of introducing surface carboxyl groups in two steps. While the silanation step appears to be reproducible, the efficiency of the succinylation step has proven to be much less reproducible, apparently due to sensitivity to variations in mixing protocols. This variability is exacerbated when the process is implemented in an automated format.

In one aspect, the succinylation step is omitted by introducing carboxyl groups directly at the silanation step (see FIG. 14). This requires a carboxylated silane, for example, the succinylated aminopropylsilanes 1502, as shown in FIG. 15, which can be readily prepared from the aminopropylsilanes and an equimolar amount of succinic anhydride. FIG. 15 illustrates a scheme of preparing carboxylated silanes according to an embodiment. This method provides a rapid, reproducible and cost-efficient process for immobilizing oligonucleotide on particles. The chemistry used for linking the oligonucleotides to the support is specific, fast and provide stable chemical bonds.

According to a further embodiment, a dipodal silane can be utilized as discussed above in section H, “General Schemes of utilizing the dipodal structure.” FIGS. 12 and 15 provide examples of a dipodal carboxylated silane, such as N,N-bis-(3-trimethoxysilylpropyl)succinamic acid (1202). 1202 can be directly silanated onto the surface 1301 as shown in FIG. 21.

FIG. 21 illustrates a scheme for attaching an oligonucleotide possessing a cleavable fluorescent tag which can be quantitated by HPLC, wherein the cleavable linker is a vicinal diol unit, Fluorophore is a fluorescein unit, and Cleavage reagent is sodium periodate.

### J. Manufacturing of Encoded Microparticles

In another embodiment, the functionalized silicon compounds are covalently attached to encoded microparticles. Examples of encoded microparticles, methods of making the same, methods for fabricating the microparticles, methods and systems for detecting microparticles, and the methods and systems for using microparticles are described in U.S. Patent Application Publication Nos. 2008/0038559, 2007/0148599, and PCT Publication No. WO 2007/081410, each of which is hereby incorporated by reference in its entirety for all purposes. In summary, the fabrication of digital, lithographically-encoded glass micro-particles involves the following exemplary process: (1) Start with a silicon wafer, (2) Deposit a silicon oxide layer, (3) Deposit a poly-silicon layer, 4) Deposit a hard-mask oxide layer, (5) Pattern the hard-mask layer (photolithographic encoding), (6) Etch the poly-silicon layer, (7) Deposit the top silicon oxide layer (encasing code in glass), (8) Pattern the particle border (define particle border), (9) Etch the oxide layer (make the border), and (10) End with removal of the silicon substrate.

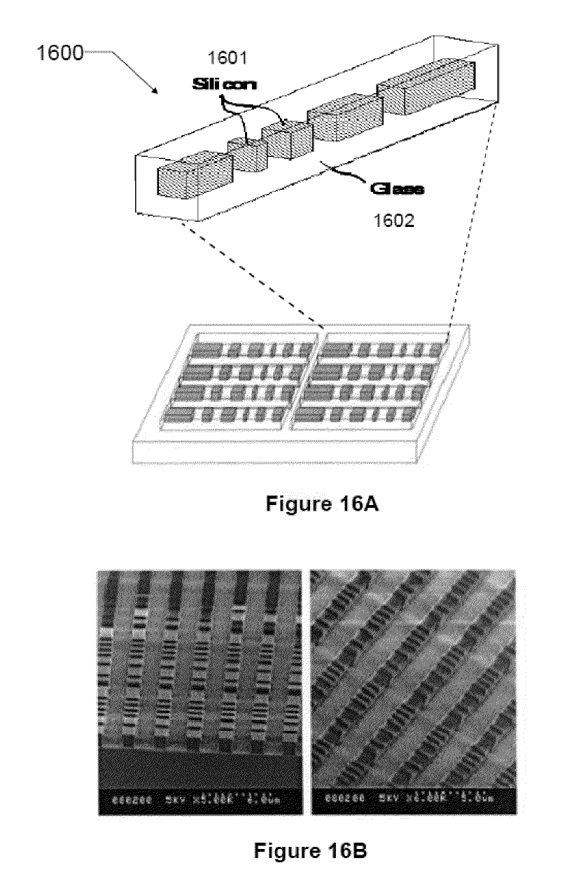

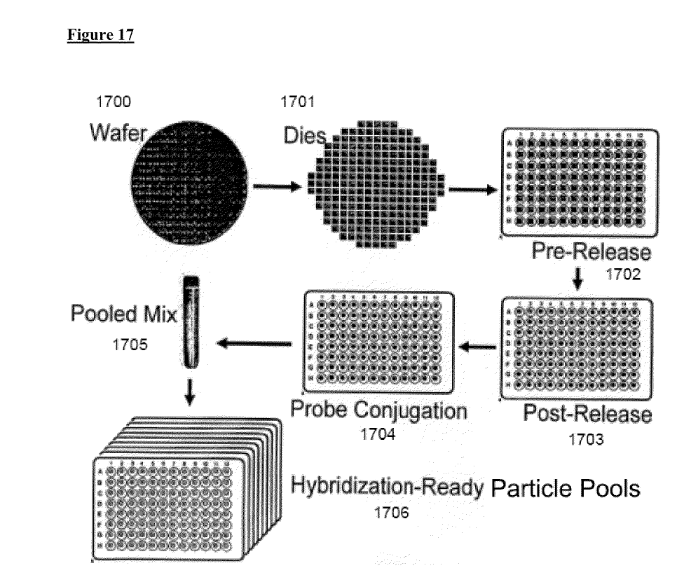

FIGS. 16A and 16B illustrate exemplary, non-limiting encoded particles (1600). FIG. 16A illustrates a schematic of individual encoded particles. FIG. 16B illustrates SEM images of the surface of an encoded particle. 1601 are silicon substrates with dies of particles encased in glass (1602).

K. Processing of Particles into Arrays

FIG. 17 illustrates an example of a schematic of work-flow for processing printed microparticles. Once the wafer is made with the printed barcodes, an exemplary process to provide mixtures of particle-probe conjugates ready for use in the hybridization-based assay is illustrated in FIG. 17. After formation of the microparticles but prior to release, the wafer 1700 can be partially cut, for example to a depth about half the wafer thickness. The wafer 1700 is then cleaned, for example with solvents and/or a strong acid (sulfuric, hydrogen peroxide combination). The cleaning is an important step as it prepares a fresh glass surface for later functionalization and biomolecule attachment. The cleaning can also be performed after the wafer 1700 is separated into individual dies 1701, or on the particles once they have been released. The silanation process may occur after the cleaning step.

The dies 1701 are placed individual wells. The particles are released in the wells at the pre-release step 1702. During the post-release step 1703, the particles are in the wells. The probes are conjugated to the particles in the wells in the probe conjugation step 1704. The particles are pooled in a tube 1705 to mix the codes. After the pool is distributed, the particle pools are ready for hybridization 1706.

### Applications

The methods and compositions disclosed herein may be used in a variety of applications. Substrates may be made having a first layer on a solid support including one or more dielectric coatings with antireflective materials and a second layer including biopolymers disposed on the first layer. In some embodiments, the substrate is substantially planar. In some embodiments, the substrate may be physically separated into regions, for example, with trenches, grooves, wells and the like. Examples of substrates include slides, beads and solid chips. The solid substrates may be, for example, biological, nonbiological, organic, inorganic, or a combination thereof, and may be in forms including particles, strands, gels, sheets, tubing, spheres, containers, capillaries, pads, slices, films, plates, and slides depending upon the intended use.

In one embodiment, high density arrays of immobilized nucleic acid probes may be formed on the substrate, and then one or more target nucleic acids comprising different target sequences may be screened for binding to the high density array of nucleic acid probes comprising a diversity of different potentially complementary probe sequences. For example, methods for light-directed synthesis of DNA arrays on glass substrates is described in McGall et al., J. Am. Chem. Soc., 119: 5081-5090 (1997), the entire disclosure of which is incorporated herein.

Methods for screening target molecules for specific binding to arrays of polymers, such as nucleic acids, immobilized on a solid substrate, are disclosed, for example, in U.S. Pat. No. 5,510,270, the disclosure of which is incorporated herein. The fabrication of arrays of polymers, such as nucleic acids, on a solid substrate, and methods of use of the arrays in different assays, are also described in: U.S. Pat. Nos. 5,774,101, 5,677,195, 5,624,711, 5,599,695, 5,445,934, 5,451,683, 5,424,186, 5,412,087, 5,405,783, 5,384,261, 5,252,743 and 5,143,854; WO 92/10092, the disclosures of each of which are incorporated herein. Accessing genetic information using high density DNA arrays is further described in Chee, Science 274: 610-614 (1996), the disclosure of which is incorporated herein by reference. The combination of photolithographic and fabrication techniques allows each probe sequence to occupy a very small site on the support. The site may be as small as a few microns or even a small molecule. Such probe arrays may be of the type known as Very Large Scale Immobilized Polymer Synthesis (VLSIPS®) arrays, as described in U.S. Pat. No. 5,631,734, the disclosure of which is incorporated herein.

In the embodiment wherein arrays of nucleic acids are immobilized on a surface, the number of nucleic acid sequences may be selected for different applications, and may be, for example, about 100 or more, or, e.g., in some embodiments, more than 10⁵ or 10⁸. In one embodiment, the surface comprises at least 100 probe nucleic acids each for example, having a different sequence, each probe contained in an area of less than about 0.1 cm², or, for example, between about 1 μm² and 10,000 μm², and each probe nucleic acid having a defined sequence and location on the surface. In one embodiment, at least 1,000 different nucleic acids are provided on the surface, wherein each nucleic acid is contained within an area less than about 10⁻³ cm², as described, for example, in U.S. Pat. No. 5,510,270, the disclosure of which is incorporated herein.

Arrays of nucleic acids for use in gene expression monitoring are described in PCT WO 97/10365, the disclosure of which is incorporated herein. In one embodiment, arrays of nucleic acid probes are immobilized on a surface, wherein the array comprises more than 100 different nucleic acids and wherein each different nucleic acid is localized in a predetermined area of the surface, and the density of the different nucleic acids is greater than about 60 different nucleic acids per 1 cm².

Arrays of nucleic acids immobilized on a surface which may be used also are described in detail in U.S. Pat. No. 5,744,305, the disclosure of which is incorporated herein. As disclosed therein, on a substrate, nucleic acids with different sequences are immobilized each in a predefined area on a surface. For example, 10, 50, 60, 100, 10³, 10⁴, 10⁵, 10⁶, 10⁷, or 10⁸ different monomer sequences may be provided on the substrate. The nucleic acids of a particular sequence are provided within a predefined region of a substrate, having a surface area for example, of about 1 cm² to 10.⁻¹⁰ cm². In some embodiments, the regions have areas of less than about 10⁻¹, 10⁻², 10⁻³, 10⁻⁴, 10⁻⁵, 10⁻⁶, 10⁻⁷, 10⁻⁸, 10.⁻⁹, or 10⁻¹⁰ cm². For example, in one embodiment, there is provided a planar, non-porous support having at least a first surface, and a plurality of different nucleic acids attached to the first surface at a density exceeding about 400 different nucleic acids/cm², wherein each of the different nucleic acids is attached to the surface of the solid support in a different predefined region, has a different determinable sequence, and is, for example, at least 4 nucleotides in length. The nucleic acids may be, for example, about 4 to 20 nucleotides in length. The number of different nucleic acids may be, for example, 1000 or more.

In the embodiment where polynucleotides of a known chemical sequence are synthesized at known locations on a substrate, and binding of a complementary nucleotide is detected, and wherein a fluorescent label is detected, detection may be implemented by directing light to relatively small and precisely known locations on the substrate. For example, in one embodiment, the substrate is placed in a microscope detection apparatus for identification of locations where binding takes place. The microscope detection apparatus includes a monochromatic or polychromatic light source for directing light at the substrate, means for detecting fluoresced light from the substrate, and means for determining a location of the fluoresced light. The means for detecting light fluoresced on the substrate may in some embodiments include a photon counter. The means for determining a location of the fluoresced light may include an x/y translation table for the substrate. Translation of the substrate and data collection are recorded and managed by an appropriately programmed digital computer, as described in U.S. Pat. No. 5,510,270, the disclosure of which is incorporated herein.

Devices for concurrently processing multiple biological chip assays may be used as described in U.S. Pat. No. 5,545,531, the disclosure of which is incorporated herein. Methods and systems for detecting a labeled marker on a sample on a solid support, wherein the labeled material emits radiation at a wavelength that is different from the excitation wavelength, which radiation is collected by collection optics and imaged onto a detector which generates an image of the sample, are disclosed in U.S. Pat. No. 5,578,832, the disclosure of which is incorporated herein. These methods permit a highly sensitive and resolved image to be obtained at high speed. Methods and apparatus for detection of fluorescently labeled materials are further described in U.S. Pat. Nos. 5,631,734 and 5,324,633, the disclosures of which are incorporated herein.

The methods and compositions described herein may be used in a range of applications including biomedical and genetic research and clinical diagnostics. Arrays of polymers such as nucleic acids may be screened for specific binding to a target, such as a complementary polynucleotide, for example, in screening studies for determination of binding affinity and in diagnostic assays. In one embodiment, sequencing of polynucleotides can be conducted, as taught in U.S. Pat. No. 5,547,839, the disclosure of which is incorporated herein. The nucleic acid arrays may be used in many other applications including detection of genetic diseases such as cystic fibrosis, diabetes, and acquired diseases such as cancer, as disclosed in U.S. patent application Ser. No. 08/143,312, the disclosure of which is incorporated herein.

Genetic mutations may be detected by sequencing by hybridization. In one embodiment, genetic markers may be sequenced and mapped using Type-IIs restriction endonucleases as disclosed in U.S. Pat. No. 5,710,000, the disclosure of which is incorporated herein.

Other applications include chip based genotyping, species identification and phenotypic characterization, as described in U.S. Pat. No. 6,228,575, filed Feb. 7, 1997, and U.S. patent application Ser. No. 08/629,031, filed Apr. 8, 1996, the disclosures of which are incorporated herein.

Gene expression may be monitored by hybridization of large numbers of mRNAs in parallel using high density arrays of nucleic acids in cells, such as in microorganisms such as yeast, as described in Lockhart et al., Nature Biotechnology, 14: 1675-1680 (1996), the disclosure of which is incorporated herein. Bacterial transcript imaging by hybridization of total RNA to nucleic acid arrays may be conducted as described in Saizieu et al., Nature Biotechnology, 16: 45-48 (1998), the disclosure of which is incorporated herein.

All publications cited herein are incorporated herein by reference in their entirety for all purposes.

### Synthesis and Use of DNA Arrays

Nucleic acid arrays that are useful include, but are not limited to, those that are commercially available from Affymetrix (Santa Clara, Calif.) under the brand name GeneChip®. There are many uses for polymers attached to solid substrates. Suitable uses include, but are not limited to, those described herein such as gene expression monitoring, profiling, library screening, genotyping and diagnostics. Methods of gene expression monitoring and profiling are described in U.S. Pat. Nos. 5,800,992, 6,013,449, 6,020,135, 6,033,860, 6,040,138, 6,177,248 and 6,309,822. Genotyping methods, and uses thereof, are disclosed in U.S. patent application Ser. No. 10/442,021 (abandoned) and U.S. Pat. Nos. 5,856,092, 6,300,063, 5,858,659, 6,284,460, 6,361,947, 6,368,799, 6,333,179, and 6,872,529. Other uses are described in U.S. Pat. Nos. 5,871,928, 5,902,723, 6,045,996, 5,541,061, and 6,197,506.

The invention may employ solid substrates. In some embodiments, the invention employs substrates for the fabrication of oligonucleotide and/or protein arrays. Methods and techniques applicable to polymer (including protein) array synthesis have been described in the art, for example, in U.S. application Ser. No. 09/536,841 (abandoned), WO 99/36760, WO 00/58516, WO 01/58593, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, which are all incorporated herein by reference in their entireties for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

General methods for the solid phase synthesis of a variety of polymer types have been previously described. Methods of synthesizing arrays of large numbers of polymer sequences, including oligonucleotides and peptides, on a single substrate have also been described. See U.S. Pat. Nos. 5,143,854 and 5,384,261 and Published PCT Application No. WO 92/10092, each of which is incorporated herein by reference in its entirety for all purposes.

As described previously, the synthesis of oligonucleotides on the surface of a substrate may be carried out using light directed methods as described in., e.g., U.S. Pat. Nos. 5,143,854 and 5,384,261 and PCT Publication No. WO 92/10092, or mechanical synthesis methods as described in U.S. Pat. Nos. 5,384,261, 6,040,193 and PCT Publication No. 93/09668, each of which is incorporated herein by reference. In particular, these light-directed or photolithographic synthesis methods involve a photolysis step and a chemistry step. The substrate surface, prepared as described herein, comprises functional groups on its surface. These functional groups are protected by photolabile protecting groups (“photoprotected”). During the photolysis step, portions of the surface of the substrate are exposed to light or other activators to activate the functional groups within those portions, e.g., to remove photoprotecting groups. The substrate is then subjected to a chemistry step in which chemical monomers that are photoprotected at least one functional group are then contacted with the surface of the substrate. These monomers bind to the activated portion of the substrate through an unprotected functional group.

In one embodiment, DNA arrays are prepared with at least one additional subsequent activation step and coupling step. In this embodiment, the at least one subsequent activation and coupling steps couple monomers to other preselected regions, which may overlap with all or part of the first region. The activation and coupling sequence at each region on the substrate determines the sequence of the polymer synthesized thereon. In one embodiment, light is shown through the photolithographic masks which are designed and selected to expose and thereby activate a first particular preselected portion of the substrate. Monomers are then coupled to all or part of this portion of the substrate. The masks used and monomers coupled in each step can be selected to produce arrays of polymers having a range of desired sequences, each sequence being coupled to a distinct spatial location on the substrate which location also dictates the polymer's sequence. In one embodiment, the photolysis steps and chemistry steps are repeated until the desired sequences have been synthesized upon the surface of the substrate.

Basic photolithographic methods are also described in U.S. Pat. No. 5,143,854, U.S. Pat. No. 5,489,678 and PCT Publication No. WO 94/10128, each of which is incorporated herein by reference in its entirety for all purposes. The surface of a substrate, modified with photosensitive protecting groups is illuminated through a photolithographic mask, yielding reactive hydroxyl groups in the illuminated regions. A selected nucleotide, typically in the form of a 3′-O-phosphoramidite-activated deoxynucleoside (protected at the 5′ hydroxyl with a photosensitive protecting group), is then presented to the surface and coupling occurs at the sites that were exposed to light. Following capping and oxidation, the substrate is rinsed and the surface is illuminated through a second mask, to expose additional hydroxyl groups for coupling. A second selected nucleotide (e.g., 5′-protected, 3′-O-phosphoramidite-activated deoxynucleoside) is presented to the surface. The selective deprotection and coupling cycles are repeated until the desired set of products is obtained. See Pease et al., Proc. Natl. Acad. Sci. (1994) 91: 5022-5026 which is hereby incorporated by reference in its entirety for all purposes. Since photolithography is used, the process can be readily miniaturized to generate high density arrays of oligonucleotide probes. Furthermore, the sequence of the oligonucleotides at each site is known.

In one embodiment, an array of polymers is synthesized on a substrate using light-directed synthesis by providing a substrate having a first layer on a solid support, said first layer including one or more stacks of dielectric materials; derivatizing said first layer by contacting said first layer with silanation reagents as described herein, and a second layer disposed on said first layer wherein said second layer includes functional groups protected with a photolabile protecting group. The method then provides for activating first selected regions on said surface of said substrate by removing said protecting groups from said functional groups in said first selected regions; coupling a first monomer to said functional groups in said first selected regions; activating second selected regions on said surface of said substrate by removing said protecting groups from said functional groups in said second selected regions; coupling a second monomer to said functional groups in said second selected regions; and repeating said activating and coupling steps to form a plurality of different polymer sequences, each of said different polymer sequences being coupled to said surface of said substrate in a different known location.

Using the above described methods, arrays may be prepared having all polymer sequences of a given length which are composed of a basis set of monomers. Such an array of oligonucleotides, made up of the basis set of four nucleotides, for example, would contain up to 4′ oligonucleotides on its surface, where n is the desired length of the oligonucleotide probe. For an array of 8 mer or 10 mer oligonucleotides, such arrays could have upwards of about 65,536 and 1,048,576 different oligonucleotides respectively. Generally, where it is desired to produce arrays having all possible polymers of length n, a simple binary masking strategy can be used, as described in U.S. Pat. No. 5,143,854.

Alternate masking strategies can produce arrays of probes which contain a subset of polymer sequences, e.g., polymers having a given subsequence of monomers, but are systematically substituted at each position with each member of the basis set of monomers. In the context of oligonucleotide probes, these alternate synthesis strategies may be used to lay down or “tile” a range of probes that are complementary to, and span the length of a given known nucleic acid segment. The tiling strategy will also include substitution of one or more individual positions within the sequence of each of the probe groups with each member of the basis set of nucleotides. These positions are termed “interogation positions”. By reading the hybridization pattern of the target nucleic acid, one can determine if and where any mutations lie in the sequence, and also determine what the specific mutation is by identifying which base is contained within the interrogation position. Tiling methods and strategies are discussed in substantial detail in U.S. Pat. No. 6,027,880, which is incorporated herein by reference in its entirety for all purposes.

Tiled arrays may be used for a variety of applications, such as identifying mutations within a known oligonucleotide sequence or “target”. Specifically, the probes on the array will have a subsequence which is complementary to a known nucleic acid sequence, but wherein at least one position in that sequence has been systematically substituted with the other three nucleotides.

### Sample Preparation for Hybridization to Arrays

In one embodiment, the invention concerns sample preparation methods, for example the preparation of a genomic DNA or cDNA sample. Prior to, or concurrent with, genotyping, the genomic sample may be amplified by a variety of mechanisms, some of which may employ PCR. (See, for example, PCR Technology: Principles and Applications for DNA Amplification, Ed. H. A. Erlich, Freeman Press, NY, N.Y., 1992; PCR Protocols: A Guide to Methods and Applications, Eds. Innis, et al., Academic Press, San Diego, Calif., 1990; Mattila et al., Nucleic Acids Res., 19:4967, 1991; Eckert et al., PCR Methods and Applications, 1:17, 1991; PCR, Eds. McPherson et al., IRL Press, Oxford, 1991; and U.S. Pat. Nos. 4,683,202, 4,683,195, 4,800,159 4,965,188, and 5,333,675, each of which is incorporated herein by reference in their entireties for all purposes. The sample may also be amplified on the array. (See, for example, U.S. Pat. No. 6,300,070 and U.S. patent application Ser. No. 09/513,300 (abandoned), all of which are incorporated herein by reference).

Other suitable sample amplification methods include the ligase chain reaction (LCR) (see, for example, Wu and Wallace, Genomics, 4:560 (1989), Landegren et al., Science, 241:1077 (1988) and Barringer et al., Gene, 89:117 (1990)), transcription amplification (Kwoh et al., Proc. Natl. Acad. Sci. USA, 86:1173 (1989) and WO 88/10315), self-sustained sequence replication (Guatelli et al., Proc. Nat. Acad. Sci. USA, 87:1874 (1990) and WO 90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909 and 5,861,245) and nucleic acid based sequence amplification (NABSA). (See also, U.S. Pat. Nos. 5,409,818, 5,554,517, and 6,063,603, each of which is incorporated herein by reference). Other amplification methods that may be used are described in, for instance, U.S. Pat. Nos. 6,582,938, 5,242,794, 5,494,810, and 4,988,617, each of which is incorporated herein by reference.

Additional methods of sample preparation and techniques for reducing the complexity of a nucleic sample are described in Dong et al., Genome Research, 11:1418 (2001), U.S. Pat. Nos. 6,361,947, 6,391,592, 6,632,611, 6,872,529 and 6,958,225, and in U.S. patent application Ser. No. 09/916,135 (abandoned).

Methods for conducting polynucleotide hybridization assays have been well developed in the art. Hybridization assay procedures and conditions will vary depending on the application and are selected in accordance with known general binding methods, including those referred to in Maniatis et al., Molecular Cloning: A Laboratory Manual, 2^{nd} Ed., Cold Spring Harbor, N.Y., (1989); Berger and Kimmel, Methods in Enzymology, Guide to Molecular Cloning Techniques, Vol. 152, Academic Press, Inc., San Diego, Calif. (1987); Young and Davism, Proc. Nat'l. Acad. Sci., 80:1194 (1983). Methods and apparatus for performing repeated and controlled hybridization reactions have been described in, for example, U.S. Pat. Nos. 5,871,928, 5,874,219, 6,045,996, 6,386,749, and 6,391,623 each of which are incorporated herein by reference.

The invention also contemplates signal detection of hybridization between ligands in certain embodiments. (See, U.S. Pat. Nos. 5,143,854, 5,578,832, 5,631,734, 5,834,758, 5,936,324, 5,981,956, 6,025,601, 6,141,096, 6,185,030, 6,201,639, 6,218,803, and 6,225,625, U.S. Patent Application Publication No. 2004/0012676 and WO 99/47964, each of which is hereby incorporated by reference in its entirety for all purposes).

The practice of the invention may also employ conventional biology methods, software and systems. Computer software products of the invention typically include, for instance, computer readable medium having computer-executable instructions for performing the logic steps of the method of the invention. Suitable computer readable medium include, but are not limited to, a floppy disk, CD-ROM/DVD/DVD-ROM, hard-disk drive, flash memory, ROM/RAM, magnetic tapes, etc. The computer executable instructions may be written in a suitable computer language or combination of several computer languages. Basic computational biology methods which may be employed in the invention are described in, for example, Setubal and Meidanis et al., Introduction to Computational Biology Methods, PWS Publishing Company, Boston, (1997); Salzberg, Searles, Kasif, (Ed.), Computational Methods in Molecular Biology, Elsevier, Amsterdam, (1998); Rashidi and Buehler, Bioinformatics Basics: Application in Biological Science and Medicine, CRC Press, London, (2000); and Ouelette and Bzevanis Bioinformatics: A Practical Guide for Analysis of Gene and Proteins, Wiley & Sons, Inc., 2^{nd} ed., (2001). See also, U.S. Pat. No. 6,420,108.

The invention may also make use of various computer program products and software for a variety of purposes, such as probe design, management of data, analysis, and instrument operation. (See U.S. Pat. Nos. 5,593,839, 5,795,716, 5,733,729, 5,974,164, 6,066,454, 6,090,555, 6,185,561, 6,188,783, 6,223,127, 6,229,911 and 6,308,170).

Additionally, the invention encompasses embodiments that may include methods for providing genetic information over networks such as the internet, as disclosed in, for instance, U.S. Patent Application Publication Nos. 2003/0097222, 2002/0183936, 2003/0100995, 2003/0120432, 2004/0002818, 2004/0126840, 2004/0049354, and U.S. Provisional Application No. 60/482,389.

### EXAMPLES

The following list of examples is provided for illustrative purposes only. While embodiments of the invention are intended to encompass these examples, it will be clear to one of skill in the art that these are non-limiting examples and that many modifications may be made to the examples while still maintaining subject matter within the scope of the invention. Therefore, these examples are non-limiting embodiments of the invention.

### Example 1

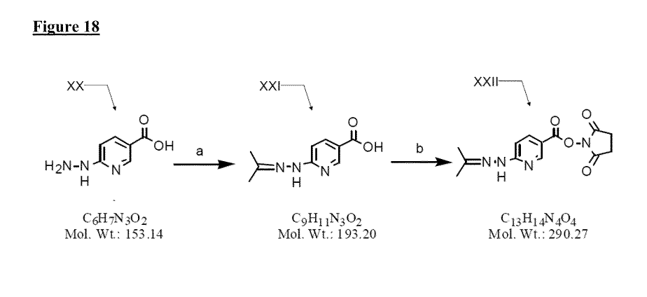

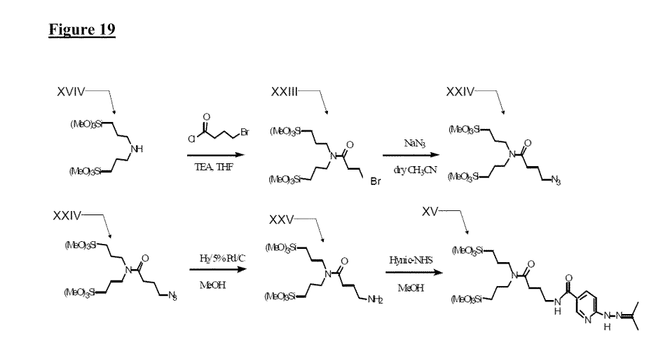

### Experimental Procedure for Synthesis of 6-(N′-Isopropylidene-hydrazino)-nicotinic acid, N-oxysuccinimidyl ester (XXII, FIG. 18)

The synthesis of 6-(N′-Isopropylidene-hydrazino)-nicotinic acid, N-oxy-succinimidyl ester (XXII) from 6-Hydrazino-nicotinic acid (XX) is shown in FIG. 18.

### Experimental Procedure

### 6-(N′-Isopropylidene-hydrazino)-nicotinic acid (XXI, FIG. 18)

Anhydrous acetone (200 mL, excess) was added to 10 g (65 mmol, 1 eq) of 6-Hydrazino-nicotinic acid (XX) and stirred at room temperature. The reaction was monitored by LCMS and after 2 hours the precipitate was collected by filtration, washed with acetone and dried under vacuum to afford 12 g (95%) of XXI as a grey solid; APCI (neg mode) m/z=192 (M−H); ¹H NMR (400 MHz, DMSO-d₆, ppm): δ 12.54 (1H, bs), 9.88 (1H, bs), 8.61 (1H, d), 8.01 (1H, dd), 7.07 (1H, d), 1.97 (3H, s), 1.94 (3H, s).

### 6-(N′-Isopropylidene-hydrazino)-nicotinic acid, N-oxysuccinimidyl ester (XXII, FIG. 18)

To 5 g (25.9 mmol, 1 eq) of XXI and 7 g (27.2 mmol, 1.1 eq) of disuccinimidylcarbonate (DSC) in 30 mL of dry acetonitirile was added slowly at room temperature 4 ml (28.3 mmol, 1.1 eq) of dry triethylamine (TEA). The reaction was monitored by LCMS and after stirring for 18 hours the precipitate was collected by filtration, washed with dry acetonitrile and dried under vacuum to afford 5.2 g (70%) of XXII as a grey solid; APCI(pos mode), m/z=291(M+H); ¹H NMR (400 MHz, DMSO-d₆, ppm): δ 10.38 (1H, s), 8.76 (1H, d), 8.11 (1H, dd), 7.17 (1H, d), 2.87 (4H, bs), 2.00 (3H, s), 1.98 (3H, s).

### Example 2

### Experimental Procedure for Synthesis of 6-Hydrazino-N,N-bis-(3-trimethoxysilanylpropyl)nicotinamide (XV, FIG. 19)

FIG. 19 illustrates the synthesis of N-{3-[Bis-(3-trimethoxysilanyl-propyl)-carbamoyl]-propyl}-6-(N′-isopropylidene-hydrazino)-nicotinamide (XV) according to an embodiment of the invention.

### Experimental Procedure

4-azidobutyryl chloride (XXIII) was prepared by the procedure oulined in Tetrahedron 1987, 43:1811-22.

### N,N-Bis-(3-(trimethoxysilyl)propyl)-4-azidobutyramide (XXIV, FIG. 19)

A solution of 4-azidobutyryl chloride (XXIII, 9.6 g; 60 mmole) in 15 ml of dry ether was added dropwise over 30 minutes to a stirring solution of N,N-Bis-(3-(trimethoxysilyl)propyl)amine (Gelest21.5 g; 60 mmole) and N,N-(diisopropyl)ethylamine (DIEA; 9.3 g; 72 mmole) in 100 ml dry ether under nitrogen at 2°-4° C. After stirring at ambient temperature for an additional 16 hr, GC analysis indicated complete conversion of the starting material. The solution was then filtered and evaporated to dryness, and the residue was taken up in 300 ml of dry ether, allowed to stand at 4° C. for 6 hr to precipitate additional byproducts, and finally filtered and evaporated again to yield 28 g of product (est. purity=92%). ¹H-NMR (CD₃OD) δ(ppm): 3.55, 3.57 (15H, 2×s); 3.27-3.40 (6H, m); 2.46 (2H, t, J=7.2 ppm); 1.87 (2H, quint, J=7.2 ppm); 1.58-1.72 (4H, m); 0.55-0.65 (4H, m).

### Bis-(trimethoxysilylpropyl)-4-aminobutyroamide (XXV, FIG. 19)

Compound XXIV (2 g, 4.4 mmol, 1 eq) was hydrogenated over 0.4 g (20% wt/wt) of 5% Pd/C (previously dried by washing with anhydrous methanol) under a balloon pressure of hydrogen with vigorous stirring at room temperature until the reaction was complete as judged by GCMS (usually in about 1 hr). The catalyst was removed by filtration and the solution was used immediately without further manipulation in the next step; GC-CIMS (CH₄), m/z 394 (M−HOMe)⁺.

### Bis-(trimethoxysilylpropyl)-6-(N′-Isopropylidene-hydrazino)-nicotinicamide

To the crude hydrogenation solution containing XXIV (assume 4.4 mmol, 1 eq) was added 1.3 g of 6-(N′-Isopropylidene-hydrazino)-nicotinic acid, N-hydroxysuccinimidyl ester (4.4 mmol, 1 eq) and the reaction was allowed to stir at room temperature for 4 hrs or until complete as determined by LCMS. The precipitate that formed was filtered and the solvent evaporated under vacuum to a viscous amber oil. The crude oil (containing some N-hydroxysuccinimide and a small amount of 6-(N′-isopropylidene-hydrazino)-nicotinic acid NHS ester (XXII) was used without further purification in silanation of particles: LCMS-APCI (pos mode), m/z 603.4 (M+H)⁺

### 6-Hydrazino-N,N-Bis-(3-trimethoxysilanylpropyl)nicotinamide silane (XV) Coupling Data

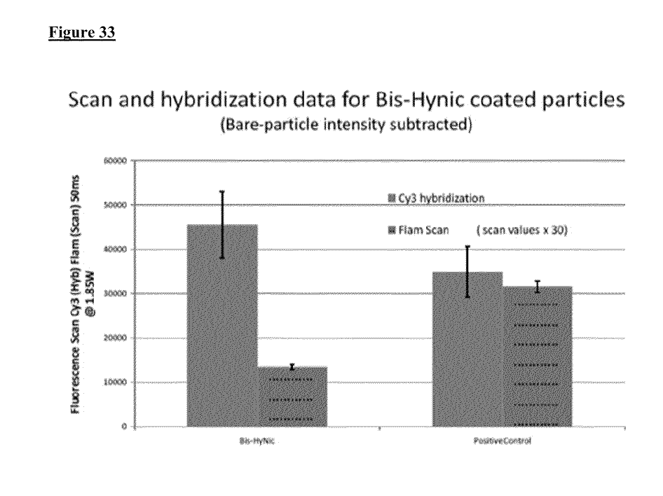

Particles were coated with the XV silane and coupled to a mixture of 20-mer oligonucleotides using the standard procedures previously discussed. The mixture of oligonucleotides were comprised of 5% of a 3′-fluorescein labeled-5′CHO— modified oligo and 95% of a 5′-CHO-modified-3′-unlabeled oligonucleotide. The fluorescence scan data was measured and then these same particles were hybridized to a complimentary Cy3-modified target oligonucleotide. The data is summarized in FIG. 33. The data indicated when compared to a positive control that oligo was sufficiently coupled as measured by the fluorescence scan intensity and efficiently hybridizes to a complimentary target sequence as indicated by the Cy3-hyb intensity.

### Example 3

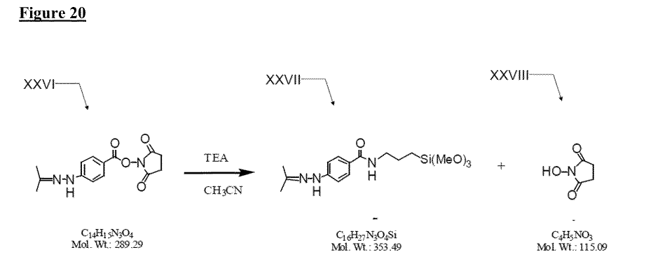

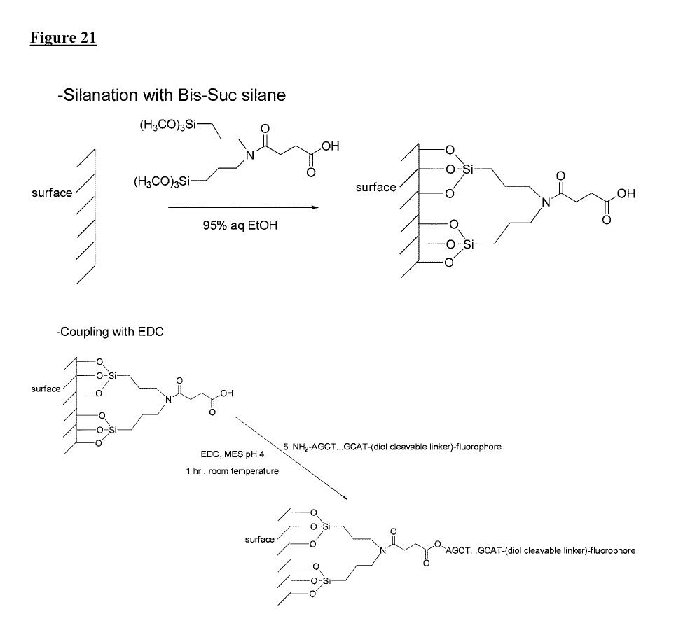

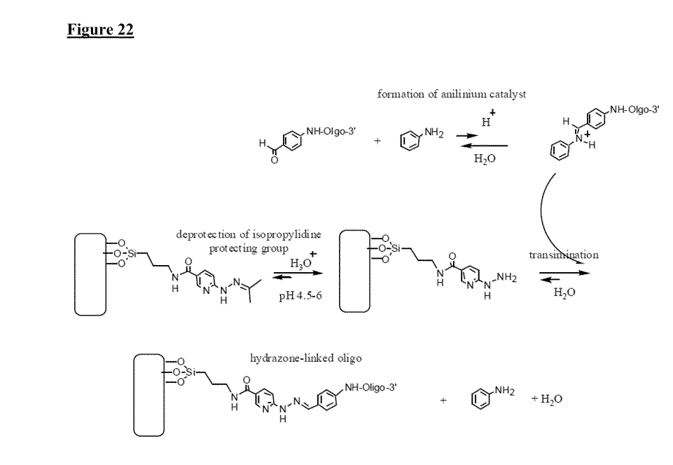

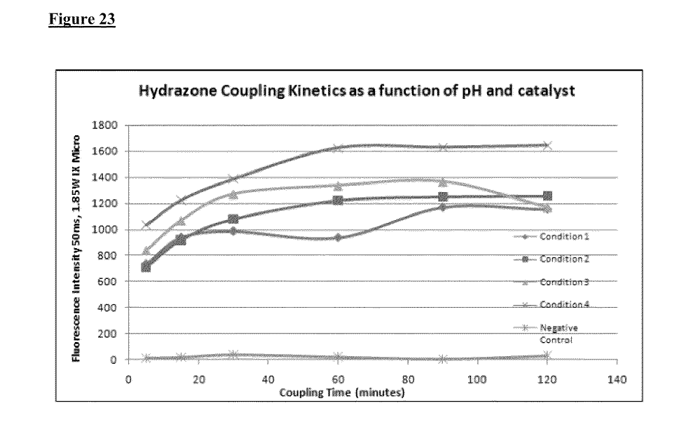

### Experimental Procedure for Synthesis of Hydrazinobenzyl Silane (XXVII, FIG. 20)

FIG. 20 illustrates the synthesis of N-trimethoxysilylpropyl-(4-N′-Isopropylidene-hydrazino)-benzamide from 4-(N′-Isopropylidene-hydrazino)-benzoic acid N-hydroxysuccinimidyl ester (XXVI).

### Experimental Procedure

To 0.5 g (1.7 mmol, 1 eq) of XXVI in 5 mL of dry acetonitrile was added at room temperature under argon 0.33 mL (1.7 mmol, 1 eq) of trimethoxysilylpropylamine. The reaction was monitored by LCMS until complete and after stirring for 4 hours the solvent was removed under vacuum affording a pale yellow oil which was determined to be a 1:1 mixture of XXVII and XXVIII.; LCMS-APCI(neg mode), m/z=352(M−H).

Generally, the silanation procedure included washing about 10⁶ particles with ethanol several times. The particles are silanated in an Eppendorf-type tube with agitation in a 1-2% solution of the silane XXVII in 95% ethanol for 1 hr at room temperature. The particles are then washed with ethanol by repeated suspension and pelleting by centrifugation and then suspended in TE buffer for storage at 4° C.

Next, the oligonucleotides are coupled. Generally, about 10⁶ silanated particles are first washed several times as above with the coupling buffer (pH range is 4.5 to 6) and then treated with a 1-2 μM solution of either a hydrazine-modified (for aldehyde-coated surface) or aldehyde-modified (for hydrazine or hydrazone-coated surface) synthetic oligonucleotide in coupling buffer for 1-2 hrs at room temperature with agitation. The particles are then washed with aqueous buffered surfactant several times and then suspended in TE buffer for storage at 4° C.

### Example 4

### Synthesis of 2-Bromo-2-methyl-N,N-bis-(3-trimethoxysilanylpropyl) propionamide

C₁₆H₃₆BrNO₇Si₂; Exact Mass: 489.1; Mol. Wt.: 490.5, m/z: 491.1 (100.0%), 489.1 (91.5%), 492.1 (28.1%), 490.1 (26.6%), 493.1 (11.1%), 494.1 (2.1%); EA: C, 39.18; H, 7.40; Br, 16.29; N, 2.86; O, 22.83; Si, 11.45

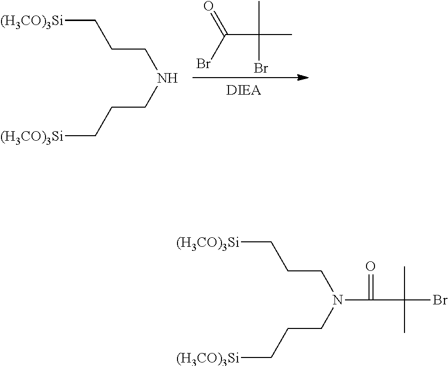

### See FIG. 11C.

A solution of 2-bromo-2-methylpropionyl bromide (37 mL; 70 g; 300 mmol) in 150 mL of dry ether was added dropwise over a period of about 45 minutes to an ice-cooled, stirring solution of N,N-Bis-(3-(trimethoxysilyl)propyl)amine (105 mL; 108 g; 300 mmol; 95%, Gelest Inc.) and N,N-(diisopropyl)ethylamine (40.6 g; 55 mL; 315 mmol) in 300 ml dry ether under nitrogen. After stirring at ambient temperature overnight, the solution was quickly and carefully filtered through a clean, dry, medium porosity vacuum filtration funnel. The filter cake was immediately washed with another 200 mL dry ether, and the combined filtrates were evaporated to dryness. The residue was then redissolved in 500 mL of dry ether, allowed to stand at 4° C. for 6 hr to precipitate additional byproducts, and finally filtered and evaporated again to yield 115 g (78%) product as an orange oil.

¹H-NMR (400 MHz; CD₃OD) δ(ppm): 3.55 (18H, s); 3.55-3.70 (2H, br m); 3.20-3.35 (2H, br m); 1.94 (6H, s); 1.55-1.85 (4H, 2×br m); 0.56-0.66 (4H, br m).

MS (APCl/MS): m/z 491.3 (M−HBr)

A general reaction scheme for the reaction set forth in this example is given in FIG. 10.

### Example 5

### Synthesis of 4-{[Bis-(3-trimethoxysilanyl-propyl)-amino]-methyl}-benzaldehyde

C₂₀H₃₇NO₇Si₂, Exact Mass: theor. m/z 459.2, Mol. Wt.: 459.7. found m/z: 459.2 (100.0%), 460.2 (33.6%), 461.2 (13.4%), 462.2 (2.9%), C, 52.26; H, 8.11; N, 3.05; O, 24.36; Si, 12.22

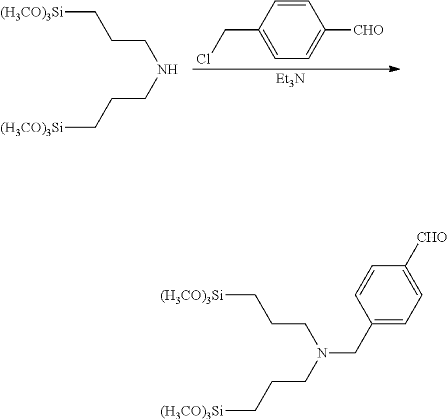

A mixture of 4-(chloromethyl)benzaldehyde (3.8 g; 24 mmole), N,N-Bis-(3-(trimethoxysilyl)-propyl)amine (95%, Gelest, 10 ml; ˜10 g; ˜28 mmole;) and triethylamine (3.0 g; 4.2 ml; 30 mmole) in 50 ml of dry acetonitrile was refluxed under Ar for 8 hours. GCMS analysis indicated disappearance of starting materials. The solvent was evaporated and the residue stirred vigorously with 150 mL dry ether and allowed to stand at room temperature for 4 hours to separate insoluble byproducts. The clear supernatant was filtered and evaporated again, and the crude product once more taken up in ether (50 mL). Dry hexanes (50 mL) was then added with vigorous stirring, and the mixture allowed to settle for 2 more hours before a final filtration and evaporation to yield 10 g (90%) of the product as a yellow oil.

¹H-NMR (400 MHz; CD₃OD) δ(ppm): 9.96 (1H, s); 7.87 (2H, d, J=8 Hz); 7.57 (2H, d, J=8 Hz); 4.85 (2H, s); 3.55 (11H, s); 3.35 (9H, s); 2.45-2.50 (4H, m); 1.53-1.62 (4H, m); 0.57-0.61 (4H, m).

¹H-NMR (400 MHz; CDCl₃) δ(ppm): 9.99 (1H, s); 7.81 (2H, d, J=8 Hz); 7.51 (2H, d, J=8 Hz); 3.63 (2H, s); 3.55 (16H, s); 2.42 (4H, t, J=7.4 Hz); 1.52-1.61 (4H, m); 0.57-0.62 (4H, m).

MS (APCI): m/z 398.2 (MH⁺−2CH₃O⁻)

### Example 6

### Synthesis of 2-[Bis-(3-trimethoxysilanyl-propyl)-amino]-ethanol

C₁₄H₃₅NO₇Si₂; Exact Mass: theor. m/z 385.2; Mol. Wt.: 385.6; m/z: 385.2 (100.0%), 386.2 (26.9%), 387.2 (11.4%), 388.2 (2.1%); EA: C, 43.61; H, 9.15; N, 3.63; O, 29.04; Si, 14.57

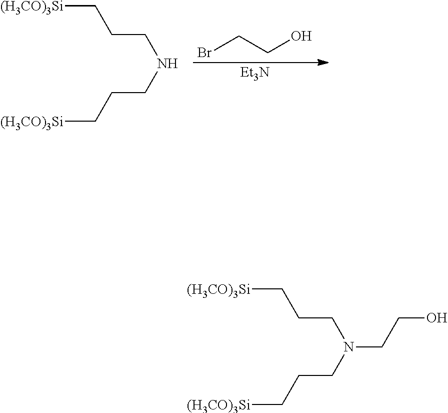

This reagent was prepared from N,N-Bis-(3-(trimethoxysilyl)-propyl)amine and 2-bromoethanol using the procedure described above in Example 5.

¹H-NMR (400 MHz; CDCl₃) δ(ppm): 3.74 (2H, t, J=5.2 Hz); 3.58 (8H, s); 3.56 (5H, s); 3.51 (4H, s); 2.55 (4H, t, J=5.2 Hz); 2.44 (2H, t, J=5.8 Hz); 1.50-1.64 (4H, m); 0.59-0.65 (2H, m); 0.53-0.58 (2H, m).

MS (EI): 354 (M−CH₃OH); 322 (M−2CH₃OH)

### Example 7

### Synthesis of N,N-Bis-(3-trimethoxysilanyl-propyl)-succinamic acid

C₁₆H₃₅NO₉Si₂; Exact Mass: theor m/z 441.2; Mol. Wt.: 441.6. found m/z: 441.2 (100.0%), 442.2 (29.2%), 443.2 (12.4%), 444.2 (2.5%); EA: C, 43.51; H, 7.99; N, 3.17; O, 32.61; Si, 12.72

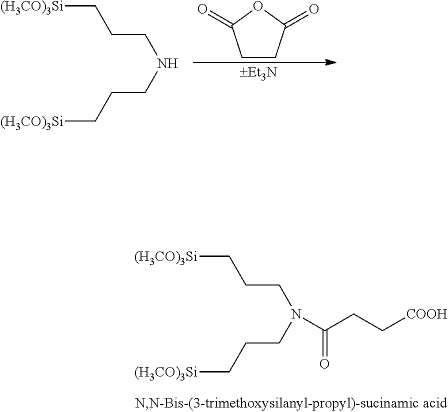

(See FIGS. 12A and 15).

Succinic anhydride (3.3 g; 33 mmole) was added in portions over 30 minutes to a vigorously stirred mixture of N,N-Bis-(3-(trimethoxysilyl)propyl)amine (Gelest, 11 g; 32 mmole) and triethylamine (3.5 g; 35 mmole) under nitrogen. The reaction was exothermic. After stirring at ambient temperature for an additional 16 hr, GC/MS analysis indicated complete conversion of the starting material. Pale yellow viscous oil.

¹H-NMR (400 MHz; CD₃OD) δ(ppm): 3.55, 3.57 (16-18H, 2×s); 3.38-3.26 (4H, m); 3.15 (6H, qrt (Et₃NH+)); 2.64 (2H, t, J=5.6); 2.56 (2H, t, J=5.6); 1.58-1.74 (4H, br m); 1.28 (9H, qrt (Et₃NH+)); 0.55-0.65 (4H, m).

¹H-NMR (400 MHz; CD₃CN) δ(ppm): 3.52, 3.50 (16-18H, 2×s); 3.28-3.18 (4H, m); 2.87 (6H, qrt (Et₃NH+)); 2.55-2.50 (2H, m); 2.40-2.35 (2H, m); 1.67-1.47 (4H, br m); 1.13 (9H, qrt (Et₃NH+)); 0.55-0.72 (4H, m). MS (APCl/neg): m/z 440.3 (M−H)

For long-term storage, the product was diluted with an equivalent volume of anhydrous methanol.

### Example 8

### Synthesis of [N,N-Bis-(3-trimethoxysilanyl-propyl)-carbamoyl]-methoxy-acetic acid

C₁₆H₃₅NO₁₀Si₂; Exact Mass: theor. 457.2; Mol. Wt.: 457.6. found m/z: 457.2 (100.0%), 458.2 (29.2%), 459.2 (12.6%), 460.2 (2.5%); EA: C, 41.99; H, 7.71; N, 3.06; O, 34.96; Si, 12.27

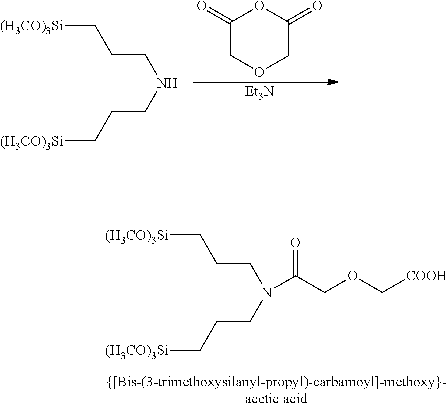

Diglycolic anhydride was reacted with N,N-Bis-(3-(trimethoxysilyl)propyl)amine using the procedure described above.

¹H-NMR (400 MHz; CDCl₃) δ(ppm): 4.28 (2H, s); 4.04 (2H, s); 3.57, 3.56 (16-18H, 2×s); 3.30-3.23 (4H, m); 2.96 (6H, qrt (Et₃NH+)); 1.69-1.59 (4H, br m); 1.23 (9H, qrt (Et₃NH+)); 0.65-0.55 (4H, m).

MS (APCl/neg): m/z 456.3 (M−H)

### Example 9

### Kinetics of Hydrazone Formation for the Coupling of DNA to Microparticles

In an attempt to characterize and optimize the hydrazone-linkage chemistry, the kinetics of coupling DNA to hydrazine silane-coated microparticles was determined for silane (XV). The dependency of rate and efficiency of coupling on reaction parameters such as oligonucleotide concentration, coupling pH and catalyst affects were investigated.

### Methods

About 10⁶ particles coated with hydrazone silane (XV in FIG. 19) were treated with 1-10 μM of a 5′-aldehyde modified oligonucleotide (5% labeled at the 3′-end with 5-fluorescein) in coupling buffer pH 4.5-6. Approximately 2×10⁵ particles were removed during the time course (because the process required centrifugation, the earliest time point taken was about 5 min.), washed and scanned for quantitation of fluorescein intensity. The scheme shown in FIG. 22 shows the likely reaction mechanism for hydrazone-based coupling of DNA to particles. The reaction includes the involvement of aniline as a catalyst.

Five reactions, including a negative control were carried out. The conditions are set out in Table 1.

TABLE 1

| | | | | Time of | |
|---|---|---|---|---|---|
| | Coupling | Buffer | Oligonucleotide | isopropylidine | |
| Condition | Buffer | pH | Concentrations | deprotection | |
| 1 | No catalyst | 6 | 1 μM | 0 | |
| 2 | 100 mM | 4.5 | 1 μM | 1 | hr |
| | anilinium | | | | |
| | acetate | | | | |
| 3 | 100 mM | 6 | 1 μM | 0 | |
| | anilinium | | | | |
| | acetate | | | | |
| 4 | 100 mM | 6 | 1 μM | 1 | hr |
| | anilinium | | | | |
| | acetate | | | | |

FIG. 23 illustrates the kinetics of hydrazone formation at 1 μM oligo concentration as a function of coupling pH, presence of catalyst and time for deprotection of isopropylidine protecting group. The kinetic curves (FIG. 23) indicated the reaction to be relatively fast with the reaction approximately 50-60% complete by the first time point allowable and reaching a plateau (saturation) in about 1.5 hr. The rates of reaction appeared not to be significantly different for changes in the pH or the presence of catalyst, although this can be a result of the fact that the rates are very fast to begin with and the rate curve not being well defined in the early stages of the reaction (FIG. 23).

However, there are some intensity differences observed favoring condition 4 where the coupling pH is 6 with the addition of 100 mM catalyst and with extended deprotection of the isopropylidine group. The increases in intensity correlate with the increase in the measured probe density data.

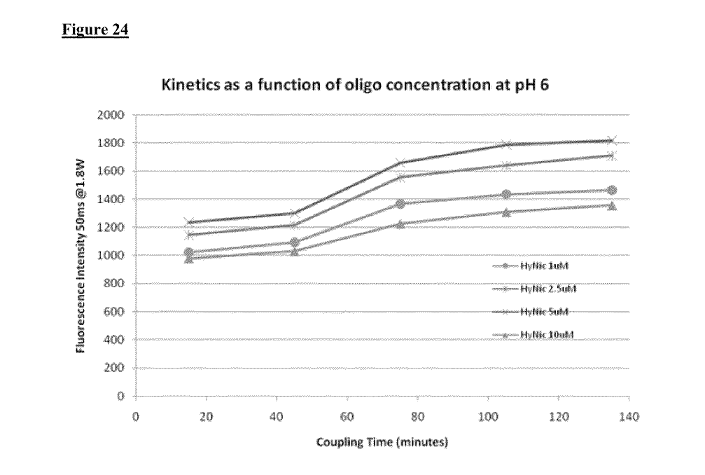

FIG. 24 illustrates the kinetics of hydrazone formation as a function of oligonucleotide concentration. In this experiment, 100 mM anilinium acetate was used as the coupling buffer (pH 6). As shown in FIG. 24, the dependency of rate and efficiency of probe coupling on oligonucleotide concentration for a fixed set of conditions was determined. The data indicated that there was not a rate dependence on the concentration in the range of 1-10 μM oligonucleotide, and that the time was essentially the same to reach saturation. Probe density data (discussed below) indicated that the oligonucleotide to hydrazine stoichiometry in the coupling reaction was at least 500:1, therefore the oligonucleotide concentration even at 1 uM is in great excess to the surface hydrazine groups and, thus, the rate may not vary significantly at these higher levels.

### Probe Density Measurements

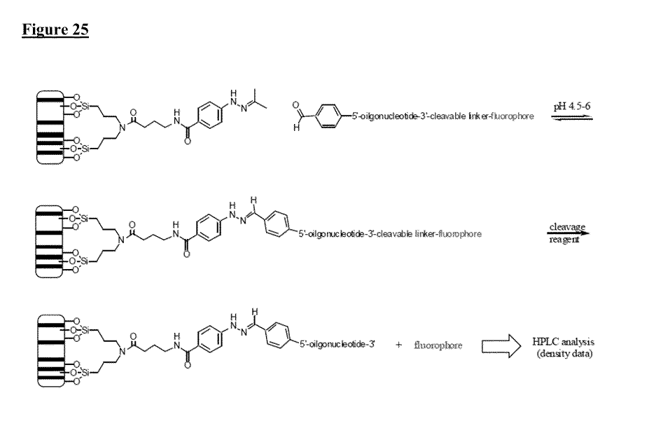

The density of oligonucleotides coupled to particles was quantitatively measured by application of a HPLC-based method for detection of a cleavable fluorescent tag. The reporter molecule was introduced by doping the coupling buffer containing the 1 μM solution of modified oligonucleotide solution with about 5% of the same oligonucleotide sequence with a fluorescent label on either the 5′ or 3′-end. Inserted between the end of the oligonucleotide and the label was a cleavable linker which was labile under near neutral conditions, and was therefore able to release the fluorescent molecule. Once the fluorescent tag was released, the amount of tag was then quantitated by HPLC using an internal concentration standard which separates from the analyte. FIG. 25 shows a typical chromatogram of about 1.5−2×10⁵ particles that have been cleaved and analyzed in the manner described. The probe density was then calculated and expressed in pmols of oligonucleotide per cm² of surface area of the particle (Table 2).

The cleavable linker can be any system which is cleaved with conditions that are orthogonal to the synthesis and deprotection conditions required for preparation of the oligonucleotide. The cleavage reagent requirement is to be benign to the silane surface, linkage or the DNA coating of the particle. One example is an 5′-aldehyde modified oligonucleotide which would be coupled to a hydrazine-modified surface to give a hydrazone linked oligonucleotide as shown in the second scheme in FIG. 21. The cleavable linker, in this case is a vicinal diol unit, which is inserted between the 3′-end of the oligonucleotide and the fluorescent tag.

TABLE 2
Probe density measurements of kinetics
experiment in FIG. 19 at 1 hr.

| | Number of Particles | Total pmol | Probe density |
|---|---|---|---|
| Condition | Cleaved | FL in Assay | (pmol/cm²) |
| 1 | 188,200 | 0.18 | 17.9 |
| 2 | 196,000 | 0.17 | 16.2 |
| 3 | 129,300 | 0.19 | 25.0 |
| 4 | 168,900 | 0.18 | 20.9 |

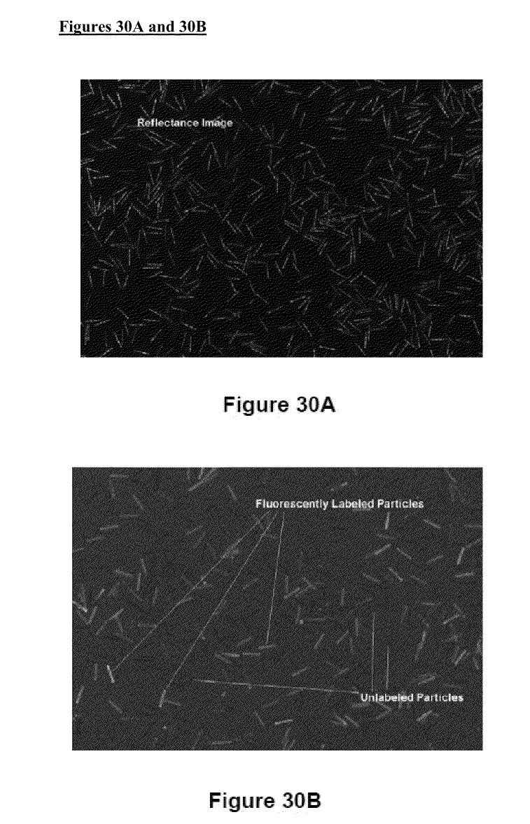

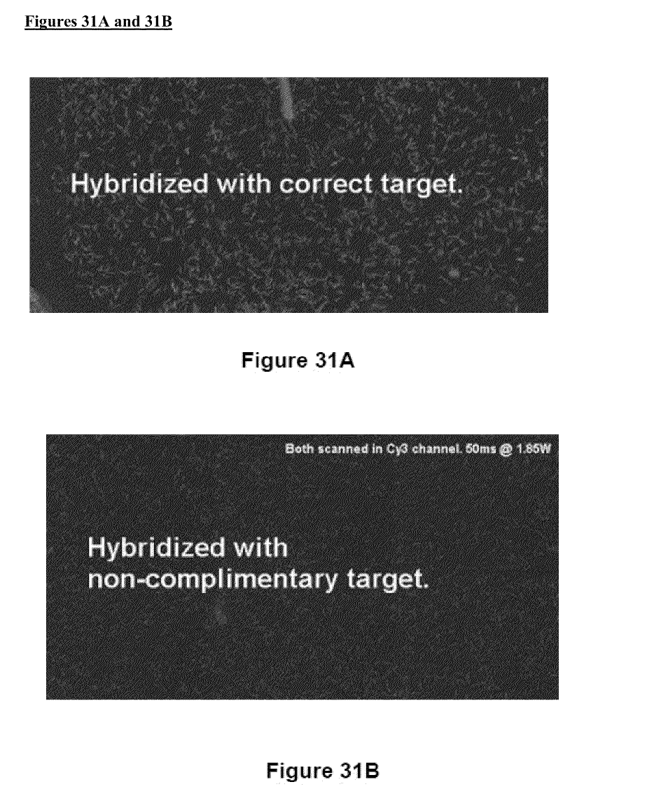

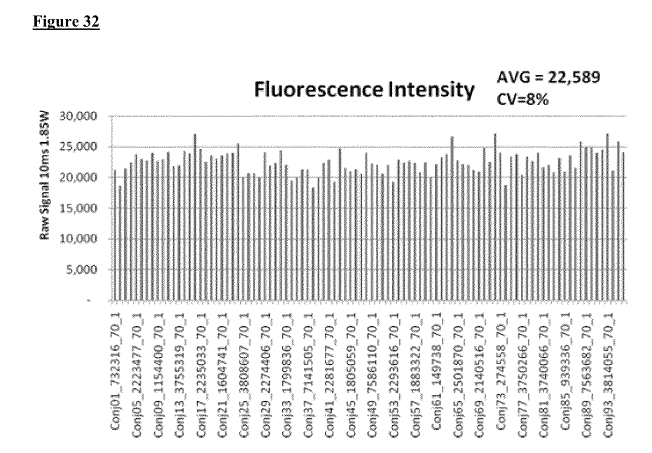

FIGS. 30A and 30B illustrate scanned images. FIG. 30A illustrates a typical image of a mixture of fluorescein-labeled DNA conjugated particles and bare particles scanned in the reflectance mode. FIG. 30B illustrates an image of fluorescein-labeled DNA conjugated particles in FIG. 30A scanned in the fluorescence mode, indicating fluorescently labeled and unlabeled particles. FIGS. 31A and 31B illustrate images of hybridization of particles. FIG. 31A illustrates an image with a Cy3-labeled complimentary target sequence. FIG. 31B illustrates an image with a Cy3-labeled non-complimentary sequence. FIG. 32 illustrates the fluorescence intensity results.

### Example 10

### General Hybridization Procedure

Conjugated particles were suspended in a hybridization buffer (any of the typical phosphate or MES-based buffers) containing about 1 nM of Cy3-labeled target DNA according to the formulation.

| | | |
|---|---|---|
| Hybridization Buffer (5X) | 20 | μL |
| Pre-labeled Synthetic Oligo mix (10 nM) | 10 | μL |
| Conjugation mix of particles | 1-5 | μL (~10⁶ particles) |
| Water | 65-69 | μL |
| TOTAL | 100 | μL |

The mixture was then heated at 95° C. for 3 min and then the hybridization was allowed to take place with agitation at the appropriate temperature (range from 37° C.-60° C.) for at least 2 hrs to 24 hrs. The hyb buffer was then removed and the particles were washed several times with a wash buffer (SSPE containing a surfactant) and stored at 4° C.

### Example 11

### General Scanning Procedure

The particles were imaged using a modified, automated Zeiss Axio Observer Z1 Inverted Fluorescence Microscope equipped for a 1536-well Nunc plate format. The following steps for imaging encoded microparticles were performed: (1) Prepared to acquire an image. About 1-5 μL of suspended bare particles (particles not silanated/coupled) were placed in a well and allowed to settle, (2) The lamp power (˜1.5 W) and exposure time (˜100 milliseconds) and magnification (40×) were set to the corresponding settings, (3) Scaned the well using the reflectance mode and then the fluorescence (fluorescein or Cy3) mode to obtain particle count and fluorescence background using custom Axio Observer Software, (4) Scaned test samples in triplicate in adjoining wells in reflectance and fluorescence mode adjusting the parameters for optimal signal, and (5) Export images and read the images using custom Part Reader Software to decode images and output assay results.

### Example 12

### Silanation and Probe Conjugation

Particles were silanated in ethanol according to the scheme in FIG. 21. Approximately 10⁶ particles were coupled to amino-modified 21-mer oligonucleotide in which 5% of the oligonucleotides bore a cleavable diol linker and fluorescein tag at the 3′-end. The concentrations of oligonucleotide were varied from 1 to 20 uM. The particles were then characterized by measurement of their fluorescence intensity, probe density, hybridization kinetics, hybridization efficiency and thermal stability.

### Example 13

### Fluorescein-Scan Intensity of Particles Conjugated with Labeled-Oligonucleotide

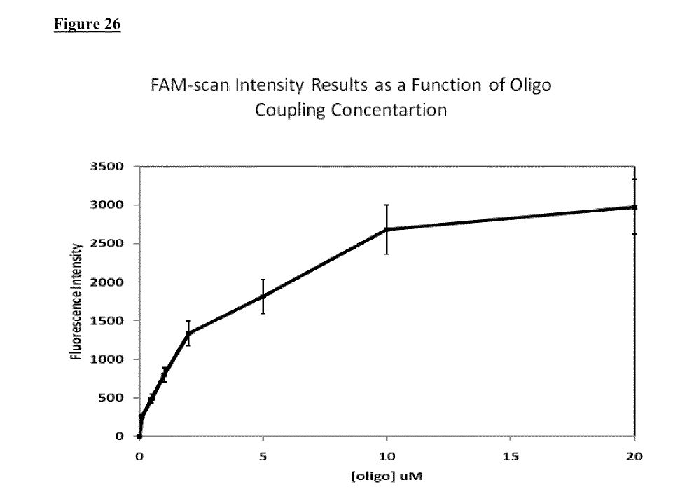

Fluorescein intensity of particles when scanned though a fluorescent microscope indicated that the intensity was exponential with increasing oligo concentration reaching a plateau level near 10 μM, as shown in FIG. 26. This is probably a consequence of probe saturation and not signal quenching since only 5% of these molecules were labeled. The same saturation-type curve was observed when the probe density was measured by an HPLC assay of released 3′-fluorescein tag from these same particles (FIG. 3), indicating that the density reached saturation at about 10 μM oligo concentration (60 corresponding to a probe density of about 10 pmols/cm²).

### Example 14

### Correlation of Fluorescence-Scan Intensity and Probe Density Measurements

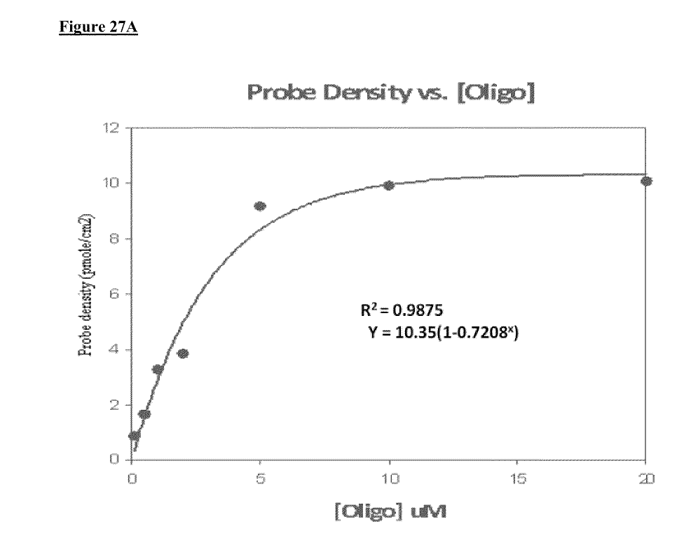

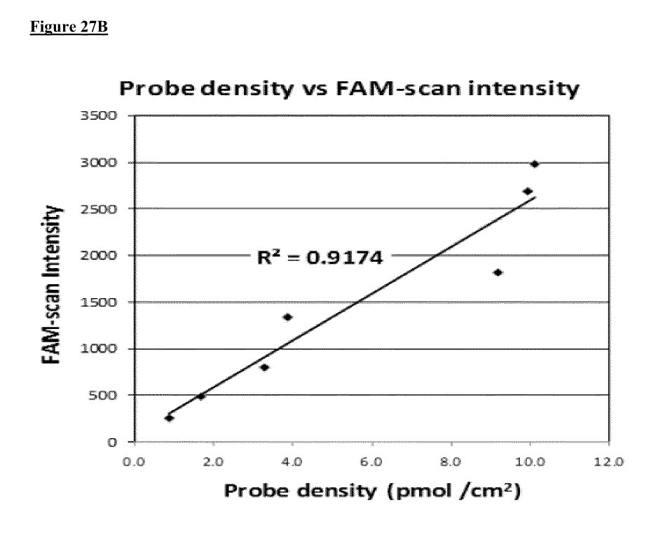

Particle probe density possesses a linear correlation with fluorescence scan intensity, as shown in FIG. 27B. (3875 FIG. 20B)

### Example 15

### Hybridization Intensity as a Function of Oligonucleotide Coupling Concentration

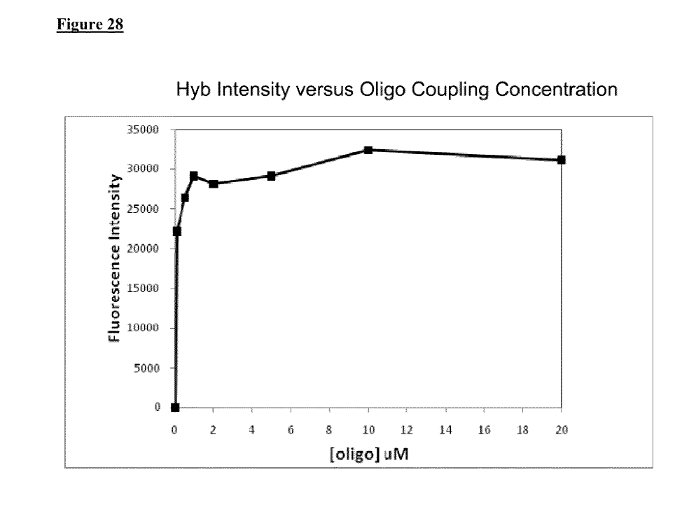

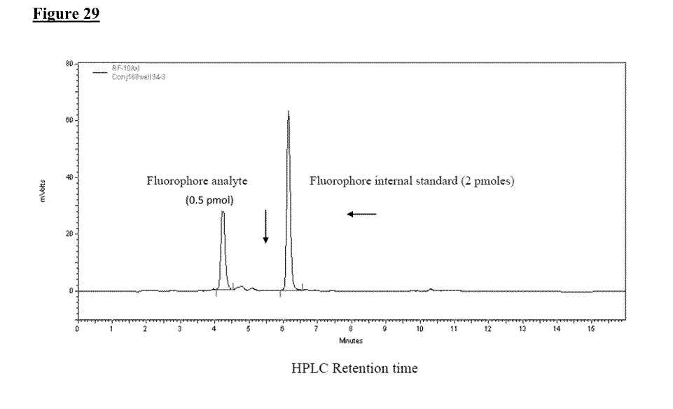

The same particles (5%-labeled with fluorescein) from FIG. 26 were hybridized to a 20 nM solution of complimentary Cy3-labeled target oligonucleotide at 40° C. in a phosphate-based buffer pH 7.4 containing SDS for 2 hrs, washed with low salt buffer and scanned on the fluorescent microscope. The plot of intensity vs. oligonucleotide coupling concentration is shown in FIG. 28. The hybridization signal saturates at ˜2 μM of coupling oligo concentration which corresponds to a probe density of about 4 pmols/cm² (according to FIG. 27A. At higher probe densities the hybridization signal increased by ˜20%.

### Example 16

### Quantitation of Hybridization Efficiency

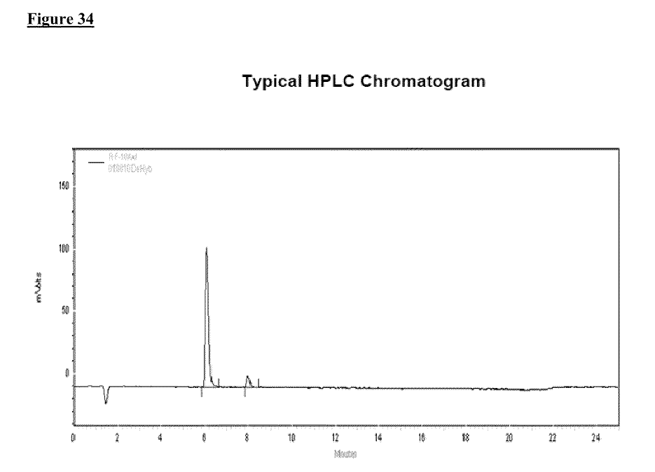

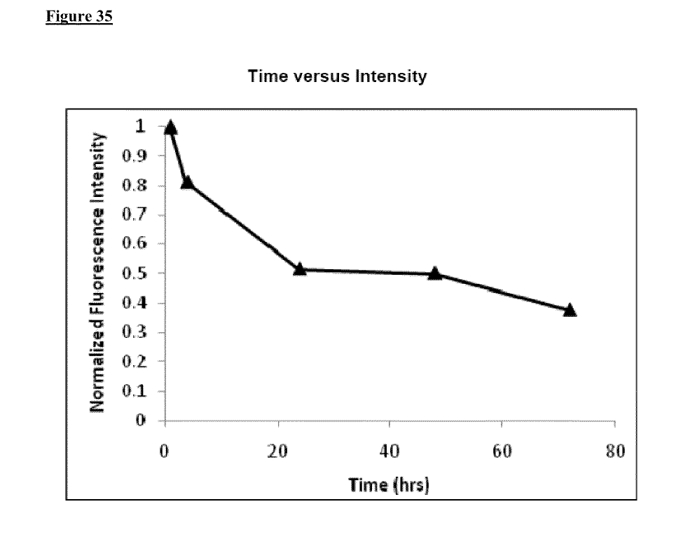

A quantitative method for determining the amount of target captured by hybridization (efficiency of hybridization) was developed based on HPLC. Particles from FIG. 28 that were hybridized to Cy3-labeled complimentary oligonucleotide target were denatured in 50% aqueous formamide at 95° C. for 5 min to release the complimentary target, the particles were then pelleted by centrifugation and the supernatant was analyzed by HPLC for quantitation of the Cy3-labeled oligonucleotide relative to a Cy3-labeled internal standard. FIG. 34 shows a typical HPLC chromatogram. In this way, knowing the probe density (FIG. 27A), the hybridization efficiency can be calculated from the ratio of target density/probe density (=0.35/7.5). In this particular case, the hybridization efficiency was ˜5%.

### Example 17

### Thermal Stability of Conjugated Particles

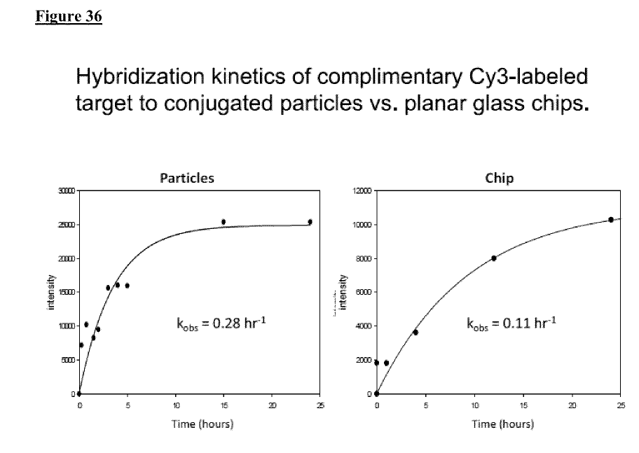

Conjugated particles used in FIG. 26 were tested for thermal stability by an accelerated degradation study in phosphate buffer at 70° C. FIG. 35 shows the loss of fluorescein-scan intensity over time. The half-life of these particles is about 15 hours at 70° C. This translates to very good stability at normal assay temperatures (Arrhenius extrapolation).

### Example 18

### Kinetics of Hybridization

The hybridization kinetics of a complimentary Cy3-labeled oligonucleotide were determined for particles prepared in FIG. 26 under the following conditions: 2 nM target concentration, 6×SSPE, pH 7.4, 40° C. The kinetics were compared to the same sequence prepared by the Affymetrix-based photolithographic process on a 2×3 in. piece of planar glass coated with bis(triethoxysilylpropyl)-3-hydroxypropylamine. The data shown in FIG. 36 indicated that the kinetics were about 2× faster than that on planar glass.

1. A method of forming an array of nucleic acids comprising:
silanating a surface of a substrate by steps comprising:
covalently attaching a plurality of functionalized silicon compounds the surface of the substrate, wherein during the silanation step at least one carboxyl group is directly introduced by silanating the surface of the substrate with a carboxylated silane compound; and
conjugating two or more oligonucleotides to the carboxylated silane compounds to form an array of nucleic acids covalently attached to the carboxylated silane compound on the surface of the substrate.

2. A method according to claim 1, wherein the substrate is a microparticle.

3. A method of functionalizing a surface comprising:
covalently attaching a functionalized silicon compound of Formula 1 to a surface of a substrate, to form a modified surface of Formula 2, wherein Formula 1 is a silicon compound having the structure: 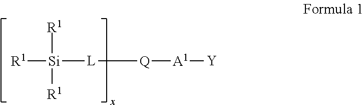
and the modified surface structure is a compound having a structure of Formula 2: 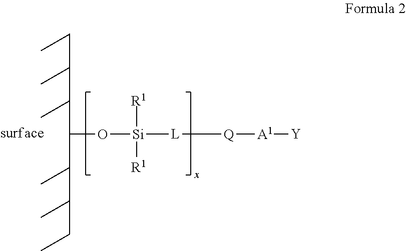
wherein, x is an integer selected from 1 to 3;
each occurrence of R¹ is independently any alkoxy, aryloxy or halogen or is a lower alkyl where at least 1 of the R¹ groups is an alkoxy or halogen;
each occurrence of L is independently a spacer group optionally comprising one or more organofunctional moieties selected from the group consisting of ether, amine, sulfide, sulfoxyl, carbonyl, thione, ester, thioester, carbonate, thiocarbonate, carbamate, thiocarbamate, amide, thioamide, urea and thiourea group;
Q is N, C₁-C₁₀ alkyl or C₁-C₁₀ substituted alkyl;
A¹ is a linking group comprising a straight chain alkyl, branched alkyl, cycloalkyl, alkenyl, alkynyl, aryl or heteroaryl, wherein A¹ optionally comprises one or more organofunctional moieties selected from the group consisting of ether, amine, sulfide, sulfonyl, sulfate, carbonyl, thione, ester, thioester, carbonate, thiocarbonate, carbamate, thiocarbamate, amide, thioamide, urea and thiourea group; and
Y is a derivatizable functional group or protected functional group selected from the group consisting of halogen, hydroxy, thiol, amine, hydrazine, aminooxy, sulfonate, sulfate, azide, carbonyl, carboxyl, carboxylate, thiocarboxyl, aldehyde, alkene, alkyne, disulfide, isocyanate, and isothiocyanate.

4. The method of claim 3, wherein x is 1 or 2 and Q is N.

5. The method of claim 4, wherein each R¹ group is methoxy.

6. The method of claim 5, wherein x is 2 and L is a C₃-C₁₀ straight chain alkyl group.

7. The method of claim 6, wherein L is a C₃ alkyl group.

8. The method of claim 7, wherein A¹ is a C₃-C₁₀ straight chain alkyl group.

9. The method of claim 8, wherein A¹ is a C₃ straight chain alkyl group further comprising a carbonyl.

10. The method of claim 9, wherein Q-A¹-Y is Q-C(═O)CH₂CH₂CH₂—Y.

11. The method of claim 10, wherein Y is a carboxyl group.

12. The method of claim 3, wherein L is a straight chain C₃-C₆ alkyl group.

13. The method of claim 3, wherein the compound of Formula 1 is selected from the
group consisting of

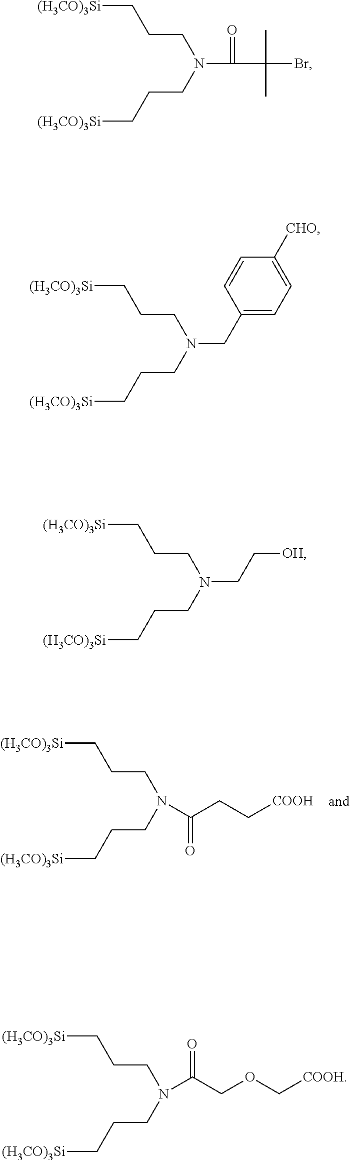

14. The method of claim 12, wherein L is a straight chain C₃ alkyl group.

15. The method of claim 3, wherein the compound of Formula 1 is represented by a compound of Formula 1(A) or 1(B):

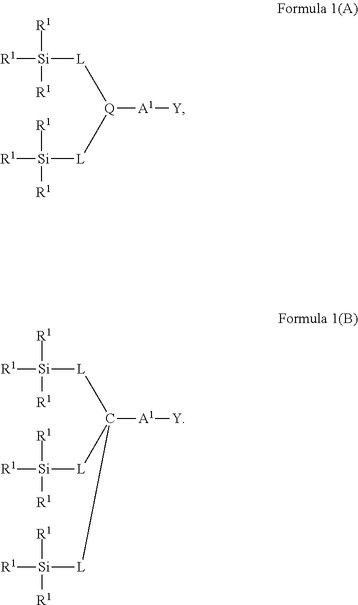

16. The method of claim 3, wherein the method further comprises:
covalently attaching a plurality of functionalized silicon compounds to the surface; and
forming an array of nucleic acids covalently attached to the functionalized silicon compounds on the surface.

17. The method of claim 3, wherein the derivatizable functional group Y is an aldehyde.

18. The method of claim 3, wherein the derivatizable functional group Y is a hydrazine or protected hydrazine.

19. The method of claim 3, wherein the derivatizable functional group Y is a carboxylate.

20. The method of claim 3, wherein the derivatizable functional group Y is an azide.

21. The method of claim 3, wherein the derivatizable functional group Y is an alkene.

22. The method of claim 3, wherein the derivatizable functional group Y is an alkyne.

23. The method of claim 3, wherein the derivatizable functional group Y is a thiol.

24. The method of claim 15, wherein the plurality of functionalized silicon compounds are covalently attached to encoded microparticles.

25. A method of functionalizing a surface comprising:
covalently attaching a plurality of functionalized silicon compounds of Formula 3 to the surface of a substrate, to form an aldehyde modified surface of Formula 4; and
reacting the surface of Formula 4 with a hydrazine-modified oligonucleotide structure of Formula 5 to produce a hydrazine-modified oligonucleotide modified surface structure of Formula 6, wherein the compound of Formula 3 has the following formula: 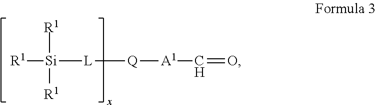
wherein the surface aldehyde structure of Formula 4 is represented by the following formula: 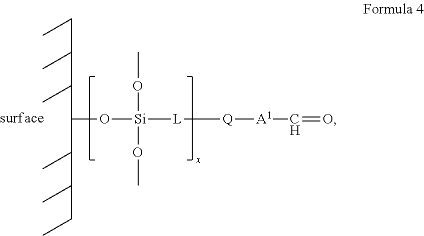
wherein the hydrazine-modified oligonucleotide structure is a compound having a structure of Formula 5: 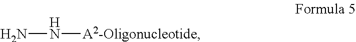
and
wherein the hydrazine-modified oligonucleotide modified surface structure is a structure of Formula 6: 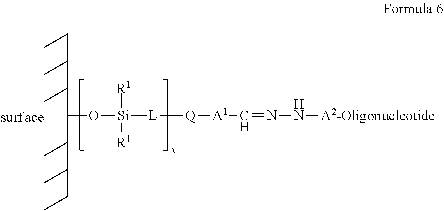
wherein, x is an integer selected from 1 to 3;
each occurrence of R¹ is independently any alkoxy, aryloxy or halogen or is a lower alkyl where at least 1 of the R¹ groups is an alkoxy or halogen;
each occurrence of L is independently a spacer group optionally comprising one or more organofunctional moieties selected from the group consisting of ether, amine, sulfide, sulfoxyl, carbonyl, thione, ester, thioester, carbonate, thiocarbonate, carbamate, thiocarbamate, amide, thioamide, urea and thiourea group;
Q is N, C₁-C₁₀ alkyl or C₁-C₁₀ substituted alkyl;
A¹ and A² are linking groups independently selected from the group consisting of a straight chain alkylor heteroalkyl, branched alkyl or heteroalkyl, cycloalkyl or heteroalkyl, alkenyl or heteroalkenyl, alkynyl or heteroalkynyl, aryl or heteroaryl, and optionally comprising organofunctional moieties selected from the group consisting of ether, amine, sulfide, sulfonyl, sulfate, carbonyl, thione, ester, thioester, carbonate, thiocarbonate, carbamate, thiocarbamate, amide, thioamide, urea and thiourea group; and
Y is a derivatizable functional group selected from the group consisting of halogen, hydroxy, thiol, amine, hydrazine, aminooxy, sulfonate, sulfate, azide, carbonyl, carboxyl, carboxylate, thiocarboxyl, aldehyde, alkene, alkyne, disulfide, isocyanate, and isothiocyanate, or a protected form thereof.

26. The method of claim 25, wherein each occurrence of R¹ is methoxy.

27. The method of claim 25, further comprising:
forming an array of nucleic acids by covalently attaching a plurality of compounds of Formula 5 to the plurality of functionalized silicon compounds on the surface.

28. The method of claim 25, wherein the derivatizable functional group Y is a hydroxyl group, activated hydroxyl group or protected hydroxyl group.

29. The method of claim 27, wherein the functionalized silicon compounds are covalently attached to encoded microparticles.

30. A method of functionalizing a surface comprising:
covalently attaching a plurality of functionalized silicon compounds of Formula 7 to the surface of a substrate to form an hydrazine modified surface of Formula 8;
reacting the surface of Formula 8 with an aldehyde-modified oligonucleotide structure of Formula 9 to produce a surface structure of Formula 10;
wherein the compound of Formula 7 has the following formula: 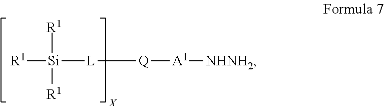
wherein the surface hydrazine structure of Formulae 8 and 8(A) are represented by the following formulae: 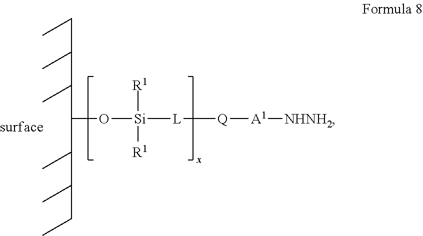
wherein the aldehyde modified oligonucleotide structure is a compound having a structure of Formula 9: 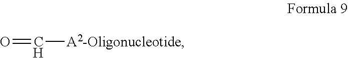
and
wherein the surface structure is a structure of Formula 10: 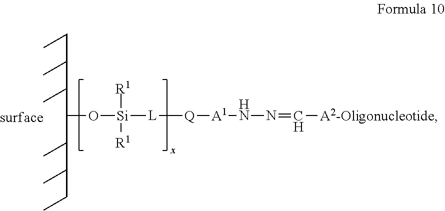
wherein, x is an integer selected from 1 to 3;
each occurrence of R¹ is independently any alkoxy, aryloxy or halogen or is a lower alkyl where at least 1 of the R¹ groups is an alkoxy or halogen;
each occurrence of L is independently a spacer group optionally comprising one or more organofunctional moieties selected from the group consisting of ether, amine, sulfide, sulfoxyl, carbonyl, thione, ester, thioester, carbonate, thiocarbonate, carbamate, thiocarbamate, amide, thioamide, urea and thiourea group; and
Q is N, C₁-C₁₀ alkyl or C₁-C₁₀ substituted alkyl;
A¹ and A² are linking groups comprising: a straight chain alkyl, branched alkyl, cycloalkyl, alkenyl, alkynyl, aryl or heteroaryl optionally comprising one or more organofunctional moieties selected from the group consisting of amine, sulfide, sulfonyl, sulfate, carbonyl, thione, ester, thioester, carbonate, thiocarbonate, carbamate, thiocarbamate, amide, thioamide, urea and thiourea group;
Y is a derivatizable functional group selected from the group consisting of halogen, hydroxy, thiol, amine, hydrazine, aminooxy, sulfonate, sulfate, azide, carbonyl, carboxyl, carboxylate, thiocarboxyl, aldehyde, alkene, alkyne, disulfide, isocyanate and isothiocyanate, or protected forms thereof.

31. The method of claim 30, wherein the method further comprises
forming an array of nucleic acids by covalently attaching a plurality of compounds of Formula 9 to the plurality of functionalized silicon compounds on the surface.

32. The method of claim 30, wherein the derivatizable functional group is a hydroxyl group, activated hydroxyl group or protected hydroxyl group.

33. The method of claim 31, wherein the functionalized silicon compounds are covalently attached to encoded microparticles.

34. A method of functionalizing a surface comprising:
covalently attaching a plurality of functionalized silicon compounds of Formula 11 to the surface of a substrate, to form an hydrazine modified surface of Formula 8 or 8(A);
reacting the surface of Formula 8 with an aldehyde-modified oligonucleotide structure of formula 9 to produce the surface structure of Formula 10;
wherein, the silicon compound of Formula 11 has the following Formula: 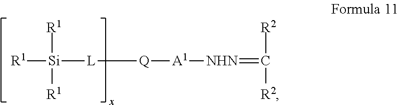
the surface hydrazine structure is a structure having a structure of Formula 8: 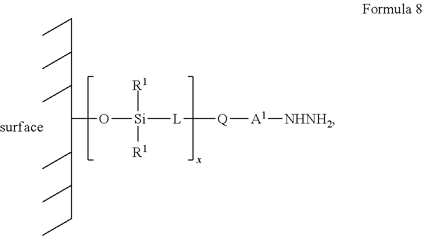
wherein the aldehyde-modified oligonucleotide structure is a compound having a structure of Formula 9: 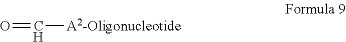
wherein the surface structure is a compound having a structure of Formula 10: 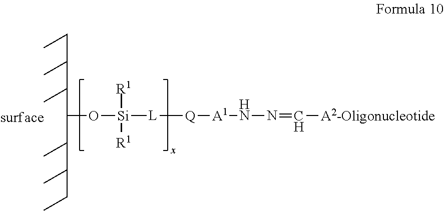
wherein, x is an integer selected from 1 to 3;
each occurrence of R¹ is independently any alkoxy, aryloxy or halogen or is a lower alkyl where at least 1 of the R¹ groups is an alkoxy or halogen;
R² and R³ are independently selected from H, alkyl, substituted alkyl, cycloalkyl and substituted cycloalkyl;
each occurrence of L is independently a spacer group optionally comprising one or more organofunctional moieties selected from the group consisting of, amine, sulfide, sulfoxyl, carbonyl, thione, ester, thioester, carbonate, thiocarbonate, carbamate, thiocarbamate, amide, thioamide, urea and thiourea group;
Q is N, C₁-C₁₀ alkyl or C₁-C₁₀ substituted alkyl;
A¹ and A² are linking groups comprising a straight chain alkyl, branched alkyl, cycloalkyl, alkenyl, alkynyl, aryl or heteroaryl, wherein each of A¹ and A² optionally comprises one or more organofunctional moieties selected from the group consisting of ether, amine, sulfide, sulfonyl, sulfate, carbonyl, thione, ester, thioester, carbonate, thiocarbonate, carbamate, thiocarbamate, amide, thioamide, urea and thiourea group;
Y is a derivatizable functional group or protected functional group selected from the group consisting of halogen, hydroxy, thiol, amine, hydrazine, aminooxy, sulfonate, sulfate, azide, carbonyl, carboxyl, carboxylate, thiocarboxyl, aldehyde, alkene, alkyne, disulfide, isocyanate, or isothiocyanate.

35. The method of claim 34, further comprising:
forming an array of nucleic acids covalently attached to the functionalized silicon compounds on the surface.

36. The method of claim 35, wherein the functionalized silicon compounds are covalently attached to encoded microparticles.

37. The method of claim 34, wherein the functionalized silicon compound is a compound of Formula 11 is: 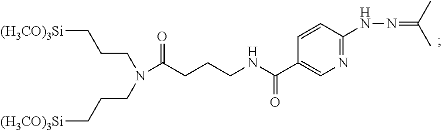
wherein x=2, Q is N—, each occurrence of R¹ is methoxy, each occurrence of L is —(CH₂)₃—,
A¹ is 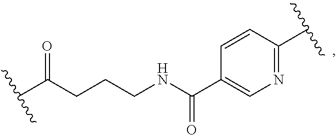
and R² and R³ are CH₃.

38. The method of claim 37, wherein the method comprises:
covalently attaching a plurality of functionalized silicon compounds to the surface; and
forming an array of nucleic acids covalently attached to the functionalized silicon compounds on the surface.

39. The method of claim 38, wherein the functionalized silicon compounds are covalently attached to encoded microparticles.

40. A compound having the Formula: 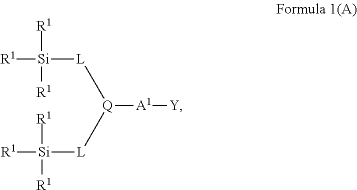
wherein, each occurrence of R¹ is independently any alkoxy, aryloxy or halogen or is a lower alkyl where at least 1 of the R¹ groups is an alkoxy or halogen;
each occurrence of L is independently a spacer group optionally comprising one or more organofunctional moieties selected from the group consisting of ether, amine, sulfide, sulfoxyl, carbonyl, thione, ester, thioester, carbonate, thiocarbonate, carbamate, thiocarbamate, amide, thioamide, urea and thiourea group;
Q is N, C₁-C₁₀ alkyl or C₁-C₁₀ substituted alkyl;
A¹ is a linking group comprising a straight chain alkyl, branched alkyl, cycloalkyl, alkenyl, alkynyl, aryl or heteroaryl, wherein A¹ optionally comprises one or more organofunctional moieties selected from the group consisting of ether, amine, sulfide, sulfonyl, sulfate, carbonyl, thione, ester, thioester, carbonate, thiocarbonate, carbamate, thiocarbamate, amide, thioamide, urea and thiourea group; and
Y is a derivatizable functional group selected from the group consisting of halogen, hydroxy, thiol, amine, hydrazine, aminooxy, sulfonate, sulfate, azide, carbonyl, carboxyl, carboxylate, thiocarboxyl, aldehyde, alkene, alkyne, disulfide, isocyanate, and isothiocyanate, or protected forms thereof.

41. The compound of claim 40, wherein A¹ comprises one or more organofunctional moieties selected from the group consisting of ether, amine, sulfide, sulfonyl, sulfate, carbonyl, thione, ester, thioester, carbonate, thiocarbonate, carbamate, thiocarbamate, amide, thioamide, urea and thiourea group.

42. The compound of claim 40, wherein A¹ comprises a carbonyl moiety.

43. The compound of claim 40, wherein each R¹ group is methoxy, each L group is propyl, Q is N, A¹ is a C₃-C₁₀ straight chain alkyl and Y is COOH.

44. The compound of claim 40, wherein each R¹ group is methoxy, each L group is propyl, Q is N, A¹ is —C(═O)CH₂CH₂—, and Y is COOH.

45. The compound of claim 41, wherein A¹ comprises a carbonyl moiety.

46. The compound of claim 42, wherein A¹ is a C₃ straight chain alkyl group comprising a carbonyl moiety.

47. The compound of claim 45, wherein A¹ is a C₃-C₁₀ alkyl group.

48. The compound of claim 47, wherein A¹ comprises a carbonyl moiety.

49. The method of claim 15, wherein the compound is a compound of Formula 1(A), and each R¹ group is methoxy, each L group is propyl, Q is N, A¹ is C₃-C₁₀ straight chain alkyl and Y is COOH.

50. The method of claim 15, wherein A¹ is a C₃-C₁₀ straight chain alkyl and comprises one or more organofunctional moieties selected from the group consisting of ether, amine, sulfide, sulfonyl, sulfate, carbonyl, thione, ester, thioester, carbonate, thiocarbonate, carbamate, thiocarbamate, amide, thioamide, urea and thiourea group.

51. The method of claim 49, wherein A¹ is a C₃ straight chain alkyl group further comprising a carbonyl.

52. The method of claim 50, wherein Q-A¹-Y is Q—C(═O)CH₂CH₂CH₂—Y.

53. The method of claim 51, wherein Y is a carboxyl group.
